(19) United States
(12) Patent Application Publication
(10) Pub. No.: US 2026/0091030 A1
(43) Pub. Date: Apr. 2, 2026

(54) HIGH-DOSE NALOXONE FORMULATION

(71) Applicant: SRI INTERNATIONAL, Menlo Park, CA (US)

(72) Inventors: Gita SHANKAR, Menlo Park, CA (US); Suresh POTHARAJU, Menlo Park, CA (US)

(21) Appl. No.: 19/111,443

(22) PCT Filed: Sep. 1, 2023
(86) PCT No.: PCT/US2023/073305

§ 371 Date: Mar. 13, 2025

Related U.S. Application Data
(60) Provisional application No. 63/407,569, filed on Sep. 16, 2022.

(51) Int. Cl. A61K 31/485; A61J 1/14; A61K 9/00; A61K 9/08; A61K 47/02; A61K 47/12; A61K 47/14; A61K 47/18

(52) U.S. Cl. CPC A61K 31/485; A61J 1/1468; A61K 9/0019; A61K 9/08; A61K 47/02; A61K 47/12; A61K 47/14; A61K 47/186

(57) ABSTRACT

Provided are formulations and methods to treat ultra-potent synthetic opioid overdose with high dose Naloxone formulations comprising naloxone hydrochloride or a pharmaceutically acceptable salt thereof, one or more preservatives, one or more buffering agents, a tonicity modifier, and optionally a pH modifier.

### CROSS-REFERENCE TO RELATED APPLICATIONS

This application claims the benefit of priority of U.S. Provisional Patent Application No. 63/407,569, filed Sep. 16, 2022, which is hereby incorporated herein by reference in its entirety.

### GOVERNMENT RIGHTS

This invention was made with U.S. Government support under Agreement No. W15QKN-16-9-1002 awarded by the U.S. Army Contracting Command-New Jersey (ACC-NJ) to the Medical CBRN Defense Consortium (MCDC). The Government has certain rights in the invention.

### BACKGROUND OF THE DISCLOSURE

### Field of the Disclosure

This disclosure relates to formulations and methods of treatment for opioid poisoning, e.g., opioid overdose. Opioid refers generally to any substance that binds to opioid receptors. The term opiate can be used to describe generally opioids that are derived from the opium poppy including morphine and codeine. Rosenblum et al., Exp. Clin. Psycholpharmacol. 16(5): 405-16 (2008). Opioids may include drugs that are synthesized from naturally occurring opiates, known as semi-synthetic opioids, such as heroin from morphine, oxycodone amongst others, as well as fully synthetic opioids such as methadone, fentanyl, propoxyphene and carfentanil. Deaths due to opioid overdoses are on the rise and remain a leading cause of death in the United States. Estimates suggest that about 92,000 people died from accidental overdose in 2020, representing a 30% increase from the prior year. Further, synthetic opioids such as fentanyl and carfentanil have been estimated to contribute to nearly 60% of the drug overdoses reported in 2020.

Although opioids have existed for thousands of years, it was only since the 19^{th} and early 20^{th} centuries that morphine in combination with scopolamine were being employed as an anesthetic. Stanley, J. Pain Symptom Manage. 7(3 Suppl): S3-S7 (1992). However, due to reports of interoperative morbidity, these anesthetic methods were largely discontinued as researchers sought new intravenous anesthetic agents. In 1953, Paul Jansen discovered dextromoramide, a 3.3-di-phenylpropylamine with potent analgesic properties. Jansen hypothesized that small variations in the structures of morphine and meperidine would lead to significant changes in analgesic activity, specifically, that increased solubility across the blood-brain barrier would lead to stronger analgesic effects. Meperidine, being 30-35 times more lipid soluble than morphine, served as a starting compound for modifications. From meperidine, phenoperidine was synthesized, the compound being approximately 20 times more potent than morphine and approximately 200 times more potent than meperidine. Beckett, J. Pharm. Pharmacol. 8(11): 848-59 (1956). Through further re-arrangement and modifications, phenoperidine lead to the formation of fentanyl, a compound hundreds of times more potent than morphine. Through the addition of a methyl ester group, fentanyl is converted to carfentanil, a compound nearly 10,000 times more potent than morphine and estimated 20-30 times more potent than fentanyl. Leen et al., Can. J. Anesth. 66(4): 414-21(2019). Such synthetic compounds exhibiting dramatic potency have been generally referred to as ultra-potent synthetic opioids.

The prevalence of ultra-potent synthetic opioids, such as fentanyl and its derivatives (e.g., carfentanil), poses a significant threat to both military and civilian populations. The synthetic opioids can often lead to the rapid onset of central nervous system and respiratory depression, and therefore, there is an unmet need to develop an effective, photo and thermostable pharmaceutical composition capable of reversing these adverse effects.

### Technical Background

Naloxone is an opioid receptor antagonist and an effective treatment for opioid poisoning that can alleviate opioid-induced adverse effects. Naloxone is a competitive antagonist of the μ, κ, and δ opioid receptors with virtually no agonist activity or production of physical dependence over a broad dose range. After exposure to synthetic opioids, molecules bind to opioid receptors, inducing effect. It is hypothesized that with a higher binding affinity, Naloxone can serve as a competitive inhibitor, dislodging the opioid from the receptor, and then replacing it. Since Naloxone has no agonist activity, the deleterious pharmacologic effects of the opioid cease. However, one shortcoming of Naloxone is that its elimination half-life is relatively short compared to that of many other opioids. Thus, after a dramatic reversal of the effects of the opioids, a subsequent return to intoxication is possible. The subsidence of Naloxone's competitive inhibitory effects allows for residual opioids in a subject to re-poison them—a phenomena termed renarcotization.

In order to treat synthetic opioid overdose as well as the occurrence of renarcotization with lower doses of Naloxone, there is a need for higher dose Naloxone formulations, since the duration of action of Naloxone HCl may be shorter than that of some opiates, the effect of the opiate may return as the effects of Naloxone HCl dissipates (i.e., renarcotization). However, there are technical issues present with high-dose Naloxone formulations. One such issue is nucleation, wherein higher concentrations of an active ingredient serve as a nexus for crystal formation. Crystal formation reduces the bioavailability of drug product and results in an unsuitable medicament. Another technical issue from high-dose Naloxone formulations is stability, both in terms of thermostability as well as photostability. Due to the global prevalence of synthetic opioids, an effective high-dose Naloxone formulation must be thermostable in temperatures at or exceeding 40° C., temperatures common in tropical and or subtropical portions of the world. Additionally, many instances of opioid poisoning occur outside of a clinical setting, meaning first responders must be able to administer a high-dose Naloxone formulation in a field setting. Therefore, an effective high-dose Naloxone formulation must be formulated and packaged to remain stable when potentially exposed to sunlight.

### SUMMARY

Described herein, in various aspects, is an aqueous pharmaceutical composition comprising: (a) naloxone hydrochloride or a pharmaceutically acceptable salt thereof, (b) one or more preservatives, (c) one or more hydroxycarboxylic acid buffering agents. (d) a tonicity modifier, and (e) optionally a pH modifier.

In various aspects, the aqueous pharmaceutical composition comprising Naloxone hydrochloride or pharmaceutically acceptable salt thereof contains Naloxone at a dose of about 3-4 mg/ml, 4.5-5 mg/ml, 5.5-6.5 mg/mL, 7-8 mg/mL, 8.5-9.5 mg/mL, 10-11 mg/mL, 11.5-12 mg/mL, 13-14 mg/mL, 14.5-15 mg/ml, 15.5-16 mg/mL, 16.5-17 mg/mL, or 17-18 mg/mL.

In various aspects, the aqueous pharmaceutical composition contains one or more preservatives that are methylparaben and propylparaben.

In various aspects, the aqueous pharmaceutical composition of Naloxone contains one or more preservatives and those preservatives are about 0.1-0.5% w/w methylparaben and about 0.1-0.5% w/w propylparaben.

In yet other various aspects, the aqueous pharmaceutical composition further comprises one or more hydroxycarboxylic acid buffering agents, and these hydroxycarboxylic acid buffering agents are trisodium citrate dihydride and citric acid monohydrate.

Furthermore, in yet another aspect, the pharmaceutical composition of Naloxone, further comprises one or more hydroxycarboxylic acid buffering agents, and these hydroxycarboxylic acid buffering agents are about 0.01-0.1% w/w trisodium citrate dihydride and about 0.01-0.5% w/w citric acid monohydrate.

In yet another aspect, the aqueous pharmaceutical composition further comprises a tonicity modifier wherein the tonicity modifier is sodium chloride, sorbitol, or mannitol.

In various other aspects, the aqueous pharmaceutical composition of Naloxone further comprises a pH modifier, and the pH modifier is sodium hydroxide or hydrochloric acid.

In various other aspects, the aqueous pharmaceutical composition of Naloxone has a pH that is about 1-2. However, in other aspects, the pH is about 2-3, 3-4, 4-5, 5-6, 6-7, 7-8, 8-9, 9-10, 10-11, 11-12, 12-13, 13-14, 14-15, or 15-16.

In other aspects, the aqueous pharmaceutical composition of Naloxone is injectable, and has an intramuscular bioavailability of about 30-40%, 40-50%, 50-60%, 60-70%, 70-80%, 80-90%, or 90-100% after injection into a subject.

In various other aspects, the aqueous pharmaceutical composition of Naloxone comprises less than about 0.05-0.15% w/w noroxymorphone as an impurity after storage for six months at 40° C.±2° C. and 75%±5% relative humidity. Examples of other Naloxone impurities include but are not limited to the following: noroxymorphone, 3-0-Allylnaloxone, 10-α-Hydroxynaloxone, 7,8-didehydronaloxone, 2,2′-bisnaloxone, and 10-β-Hydroxynaloxone.

In various other aspects, the aqueous pharmaceutical composition of Naloxone comprises less than about 0.10-0.14% w/w noroxymorphone as an impurity after storage for thirty six months at 25° C.±2° C./60%±5% relative humidity. Examples of other Naloxone impurities include but are not limited to the following: noroxymorphone, 3-0-Allylnaloxone, 10-α-Hydroxynaloxone, 7,8-didehydronaloxone, 2,2′-bisnaloxone, and 10-β-Hydroxynaloxone.

In various other aspects, the aqueous pharmaceutical composition of Naloxone, comprises less than about 0.01-0.0% w/w noroxymorphone as an impurity after storage for thirty six months at 5° C.±3° C. Examples of other Naloxone impurities include but are not limited to the following: noroxymorphone, 3-O-Allylnaloxone, 10-α-Hydroxynaloxone, 7,8-didehydronaloxone, 2,2′-bisnaloxone, and 10-β-Hydroxynaloxone.

In other aspects, the aqueous pharmaceutical composition of Naloxone, is packaged in a single-use vial.

In other aspects, the aqueous pharmaceutical composition of Naloxone, is packaged in a multi-use vial allowing for up to nine repeat doses or about 3-4 mg, 4.5-5 mg, 5.5-6.5 mg, 7-8 mg, 8.5-9.5 mg, 10-11 mg, 11.5-12 mg, 13-14 mg, 14.5-15 mg, 15.5-16 mg, 16.5-17 mg, or 17-18 mg per dose.

In yet another aspect, the aqueous pharmaceutical composition of Naloxone, is further packaged in a clear vial.

In yet another various aspect, the aqueous pharmaceutical composition of Naloxone, is further packaged in an amber vial.

In yet another aspect, the aqueous pharmaceutical composition of Naloxone, is further packaged in a clear vial or amber vial under an atmosphere of nitrogen.

In another aspect, the aqueous pharmaceutical composition of Naloxone is packaged in an opaque container so as to protect from photo-degradation. This opaque container may be made of cellulosic compounds such as cardboard, cardstock, wax-coated paper, recycled paper, or other paper materials. In yet other aspects, the opaque container may be constructed out of polymers such as plastics such as polypropylene, polyethylene, high density polyethylene, low density polyethylene, polycarbonate, polyethylene terephthalate, polystyrene, acrylics such as acrylonitrile butadiene, polyvinyl chloride, polyurethane, nylon, and other such polymers.

In yet another aspect, the aqueous pharmaceutical composition comprises: (a) naloxone hydrochloride or a pharmaceutically acceptable salt thereof, (b) methylparaben, (c) propylparaben, (d) trisodium citrate dihydride, (e) citric acid monohydrate, (f) a tonicity modifier, and (g) optionally a pH modifier, wherein the aqueous pharmaceutical composition has a pH of about 3.0.

In yet another aspect, the aqueous pharmaceutical composition of Naloxone, contains naloxone hydrochloride or pharmaceutically acceptable salt thereof at a dose of about 8.5-9.5 mg/mL, 10-11 mg/ml, 11.5-12 mg/mL, 12-13 mg/mL, 13-14 mg/mL, 14.5-15 mg/mL, 15.5-16 mg/mL, 16.5-17 mg/mL, or 17-18 mg/mL.

In yet another aspect, the aqueous pharmaceutical composition of Naloxone, contains a tonicity modifier that is sodium chloride, sorbitol, or mannitol.

In yet a further aspect, the aqueous pharmaceutical composition of Naloxone, contains a pH modifier that is sodium hydroxide or hydrochloric acid.

In yet another aspect, the aqueous pharmaceutical composition of Naloxone, is injectable and results in intramuscular bioavailability of about 30-40%, 40-50%, 50-60%, 60-70%, 70-80%, 80-90%, or 90-100% after injection into a subject.

In yet another aspect, the aqueous pharmaceutical composition of Naloxone, comprises less than about 0.05-0.15% w/w noroxymorphone as an impurity after storage for six months at 40° C.±2° C. and 75%±5% relative humidity. In various other aspects, the aqueous pharmaceutical composition of Naloxone comprises less than about 0.10-0.14% w/w noroxymorphone as an impurity after storage for thirty six months at 25° C.±2° C./60%±5% relative humidity.

In yet another aspect, the aqueous pharmaceutical composition of Naloxone, is packaged in a single-use vial.

In yet another aspect, the aqueous pharmaceutical composition of Naloxone, is packaged in a multi-use vial allowing for up to nine repeat doses of 7-8 mg, 8.5-9.5 mg, 10-11 mg, 11.5-12 mg, 13-14 mg, 14.5-15 mg, 15.5-16 mg, 16.5-17 mg, or 17-18 mg per dose.

In another aspect, the aqueous pharmaceutical composition is packaged in a clear vial.

In another aspect, the aqueous pharmaceutical composition of Naloxone is packaged in an opaque container so as to protect from photo-degradation. This opaque container may be made of cellulosic compounds such as cardboard, cardstock, wax-coated paper, recycled paper, or other paper materials. In yet other aspects, the opaque container may be constructed out of polymers such as plastics such as polypropylene, polyethylene, high density polyethylene, low density polyethylene, polycarbonate, polyethylene terephthalate, polystyrene, acrylics such as acrylonitrile butadiene, polyvinyl chloride, polyurethane, nylon, and other such polymers.

In yet another aspect, the Naloxone formulation further comprises a parenteral pharmaceutical composition comprising: (a) naloxone hydrochloride or a pharmaceutically acceptable salt thereof, (b) methylparaben, (c) propylparaben, (d) trisodium citrate dihydride. (e) citric acid monohydrate, (f) sodium chloride, and (g) optionally a pH modifier; wherein the parenteral pharmaceutical composition has a pH of about 3.0; and wherein the naloxone hydrochloride or pharmaceutically acceptable salt thereof is present at a dose of about 10 mg/mL.

The parenteral pharmaceutical composition of Naloxone, may further comprise a pH modifier wherein the pH modifier is sodium hydroxide or hydrochloric acid.

In still other variations, the pharmaceutical composition of Naloxone may be a parenteral composition, wherein the parenteral composition is injectable and results in intramuscular bioavailability of about 30-40%, 40-50%, 50-60%, 60-70%, 70-80%, 80-90%, or 90-100% after injection into a subject.

In yet another aspect, the aqueous pharmaceutical composition of Naloxone, comprises less than about 0.05-0.15% w/w noroxymorphone as an impurity after storage for six months at 40° C.±2° C. and 75%±5% relative humidity.

In yet another aspect, the aqueous pharmaceutical composition of Naloxone, is packaged in a single-use vial.

In yet another aspect, the aqueous pharmaceutical composition of Naloxone, is packaged in a multi-use vial allowing for up to nine repeat doses of 7-8 mg, 8.5-9.5 mg, 10-11 mg, 11.5-12 mg, 13-14 mg, 14.5-15 mg, 15.5-16 mg, 16.5-17 mg, or 17-18 mg per dose.

In other aspects, the aqueous pharmaceutical composition of Naloxone is packaged in a clear vial.

In another aspect, the aqueous pharmaceutical composition of Naloxone is packaged in an opaque container so as to protect from photo-degradation. This opaque container may be made of cellulosic compounds such as cardboard, cardstock, wax-coated paper, recycled paper, or other paper materials. In yet other aspects, the opaque container may be constructed out of polymers such as plastics such as polypropylene, polyethylene, high density polyethylene, low density polyethylene, polycarbonate, polyethylene terephthalate, polystyrene, acrylics such as acrylonitrile butadiene, polyvinyl chloride, polyurethane, nylon, and other such polymers.

In yet another aspect, this disclosure relates to a method of treating ultra-potent synthetic opioid overdose by administering up to nine repeat doses of a parenteral pharmaceutical composition, wherein the parenteral pharmaceutical comprises: (a) about 1.0% w/w naloxone hydrochloride or a pharmaceutically acceptable salt thereof, (b) about 0.18% w/w methylparaben, (c) about 0.02% w/w propylparaben, (d) about 0.05% w/w trisodium citrate dihydride, (e) about 0.17% w/w citric acid monohydrate, (f) about 0.9% w/w sodium chloride, and (g) optionally a pH modifier of sodium hydroxide or hydrochloric acid; wherein the parenteral pharmaceutical composition has a pH of about 3.0; and wherein the naloxone hydrochloride or pharmaceutically acceptable salt thereof is present at a dose of about 10 mg/mL.

In yet another aspect, the method of treating ultra-potent synthetic opioid overdose by administering up to nine repeat doses of a parenteral pharmaceutical composition further comprises a method wherein the parenteral pharmaceutical composition is administered intravenously.

In yet another aspect, the method of treating ultra-potent synthetic opioid overdose by administering up to nine repeat doses of a parenteral pharmaceutical composition further comprises a method wherein the parenteral pharmaceutical composition is administered by intramuscular injection.

In yet another aspect, the method of treating ultra-potent synthetic opioid overdose by administering up to nine repeat doses of a parenteral pharmaceutical composition further comprises a method wherein the parenteral pharmaceutical composition is administered by subcutaneous injection.

In yet another aspect, this disclosure also relates to a parenteral pharmaceutical composition for the treatment of ultra-potent synthetic opioid overdose, wherein the parenteral pharmaceutical comprises: (a) about 1.0% w/W naloxone hydrochloride or a pharmaceutically acceptable salt thereof, (b) about 0.18% w/w methylparaben, (c) about 0.02% w/w propylparaben, (d) about 0.05% w/w trisodium citrate dihydride, (e) about 0.17% w/w citric acid monohydrate, (f) about 0.9% w/w sodium chloride, and (g) optionally a pH modifier of sodium hydroxide or hydrochloric acid; wherein the parenteral pharmaceutical composition has a pH of about 3.0; and wherein the naloxone hydrochloride or pharmaceutically acceptable salt thereof is present at a dose of about 10 mg/mL.

In yet another aspect, this disclosure also relates to the use of a parenteral pharmaceutical composition for the manufacture of a medicament for treating ultra-potent synthetic opioid overdose, wherein the parenteral pharmaceutical comprises: (a) about 1.0% w/w naloxone hydrochloride or a pharmaceutically acceptable salt thereof, (b) about 0.18% w/w methylparaben, (c) about 0.02% w/w propylparaben, (d) about 0.05% w/w trisodium citrate dihydride, (e) about 0.17% w/w citric acid monohydrate, (f) about 0.9% w/w sodium chloride, and (g) optionally a pH modifier of sodium hydroxide or hydrochloric acid; wherein the parenteral pharmaceutical composition has a pH of about 3.0; and wherein the naloxone hydrochloride or pharmaceutically acceptable salt thereof is present at a dose of about 10 mg/mL.

In yet another aspect, this disclosure also relates to a kit for administering the aqueous pharmaceutical composition of Naloxone, the kit comprising: (a) a testing apparatus to determine if a subject is suffering from opioid poisoning; (b) an autoinjector, preloaded with a therapeutic amount of the aqueous pharmaceutical composition of claim 1; (c) a product use pamphlet, describing how to administer and interpret results from the testing apparatus and how to actuate the autoinjector; (d) an opaque container to house the testing apparatus, the autoinjector, and the product use pamphlet, wherein a subject suffering from opioid poisoning or a first responder treating a subject suffering from opioid poisoning can open the opaque container, read the product use pamphlet, administer the testing apparatus, and upon confirming that the subject is suffering from opioid poisoning, actuate the autoinjector to deliver the pharmaceutical composition of Naloxone to the subject.

In yet another aspect, the Kit may further have an autoinjector, preloaded with a therapeutic amount of the aqueous pharmaceutical composition of Naloxone where the pharmaceutical composition of Naloxone further comprises (a) about 1.0% w/w naloxone hydrochloride or a pharmaceutically acceptable salt thereof; (b) about 0.18% w/w methylparaben; (c) about 0.02% w/w propylparaben; (d) about 0.05% w/w trisodium citrate dihydride; (e) about 0.17% w/w citric acid monohydrate; (f) about 0.9% w/w sodium chloride; and (g) optionally a pH modifier of sodium hydroxide or hydrochloric acid; wherein the aqueous pharmaceutical composition has a pH of about 3.0; and wherein the naloxone hydrochloride or pharmaceutically acceptable salt thereof is present at a dose of about 10 mg/mL.

### BRIEF DESCRIPTION OF THE DRAWINGS

FIG. 1 is a graph showing hypothetical Naloxone concentration in the central nervous system comparing high-dose naloxone verses traditional doses of Naloxone.

FIG. 2 is a graph showing the solubility of Naloxone relative to increasing pH.

FIG. 3 is a graph that depicts the concentration of Naloxone in various buffers at t=0 and t=7. The t=7 represents samples stored at either 40° C. or 60° C. for up to 7 days.

FIG. 4 is a graph that depicts the change in pH of Naloxone in various buffers t=0 and t=7. The t=7 represents samples stored at either 40° C. or 60° C. for up to 7 days.

FIG. 5 is a graph that depicts pH-stability of Naloxone after 7 days at room temperature by showing Naloxone and Noroxymorphone concentrations in various buffers assayed by HPLC. Temperature and duration of sample storage are at ambient temperature for 7 days.

FIG. 6 is a graph that depicts pH-stability of Naloxone after 7 days at 40° C. and 75% relative humidity by showing Naloxone and Noroxymorphone concentrations in various buffers assayed by HPLC. Temperature and duration of sample storage are at 40° C. and 75% relative humidity for 7 days.

FIG. 7 is a graph that depicts pH-stability of Naloxone after 7 days at 60° C. by showing Naloxone and Noroxymorphone concentrations in various buffers assayed by HPLC. Temperature and duration of sample storage are at 60° C. and humidity was not maintained for 7 days.

FIG. 8 is an HPLC chromatogram of naloxone degradation in a citrate buffer with pH 3.0 after storage for 7 days.

FIG. 9 is an HPLC chromatogram of naloxone degradation in a citrate buffer with pH 4.6 after storage for 7 days.

FIG. 10 is an HPLC chromatogram of naloxone degradation in a citrate buffer with pH 5.2 after storage for 7 days.

FIG. 11 is an HPLC chromatogram of naloxone degradation in a citrate buffer with pH 5.0 after storage for 7 days.

FIG. 12 is an HPLC chromatogram of naloxone degradation in a potassium phosphate buffer with pH 6.5 after storage for 7 days.

FIG. 13 is an HPLC chromatogram of naloxone degradation in a sodium phosphate buffer with pH 7.4 after storage for 7 days.

FIG. 14 is an HPLC chromatogram of naloxone degradation in a glycine-sodium hydroxide buffer with pH 10.0 after storage for 7 days.

FIG. 15 is an HPLC chromatogram of naloxone degradation in a sodium carbonate-bicarbonate buffer with pH 10.6 after storage for 7 days.

FIG. 16 is a graph showing pH fluctuations in samples under nitrogen gas overlay, specifically change in pH of Naloxone solutions in water and buffers at pH 3.0 and 4.6 on Day 0 and Day 7. The Day 7 represents samples stored at either room temperature, 40° C. and 75% relative humidity or 60° C. or 25° C. and 60% relative humidity (in amber vials) for up to 7 days.

FIG. 17 is an HPLC chromatogram of naloxone degradation in a non-buffered solution at pH 4.6 at 60° C. after storage for 7 days.

FIG. 18 is an HPLC chromatogram of naloxone degradation in a acetic acid/sodium acetate buffer solution at pH 4.6 at 60° C. after storage for 7 days.

FIG. 19 is a comparison of HPLC chromatograms of naloxone degradation in a non-buffered solution at pH 4.6 at room temperature, 40° C. and 75% relative humidity, and 60° C. after storage for 5 weeks.

FIG. 20 is a comparison of HPLC chromatograms of naloxone degradation in an acetate-buffered solution at pH 4.6 at room temperature, 40° C. and 75% relative humidity, and 60° C. after storage for 5 weeks.

FIG. 21 is a graph showing pH fluctuations of Naloxone with different buffing agents and stored at 40° C. and 75% relative humidity for 5 weeks.

FIG. 22 is a graph showing pH fluctuations of Naloxone with different buffing agents and stored at 60° C. for 5 weeks.

FIG. 23 is a graph showing pH fluctuations of Naloxone at a dose of 15 mg/mL containing sodium chloride as a tonicity modifying agent stored at 60° C. for 2 and 4 weeks.

FIG. 24 is a graph showing pH fluctuations of Naloxone at a dose of 15 mg/mL containing benzalkonium chloride, methylparaben, and propylparaben as preservatives stored at 60° C. for 2 weeks.

FIG. 25 is a comparison of HPLC chromatograms of naloxone degradation with benzalkonium chloride as a preservative coupled with various buffering agents at pHs of 3.0 and 4.6 at 60° C. after storage for 2 weeks.

FIG. 26 is a comparison of HPLC chromatograms of naloxone degradation with benzalkonium chloride as a preservative coupled with various buffering agents at pHs of 3.0 and 4.6 at 60° C. after storage for 4 weeks.

FIG. 27 is a graph showing the fluctuations of pH of Naloxone formulations stored at 60° C. for 2 weeks in various buffer systems with changing Naloxone dosing.

FIG. 28 is a graph showing fluctuations of pH of Naloxone formulations stored at 60° C. for two weeks comparing citrate buffer to acetate buffer.

FIG. 29 is a graph showing the osmolality of Naloxone formulations in various buffers after storing at 60° C. for 2 weeks.

FIG. 30 is a graph showing the osmolality of Naloxone formulations in citrate and acetate buffers at pH 3.0 and 4.6 respectively, after storing at 60° C. for 2 weeks.

FIG. 31 is a graph showing the viscosity of Naloxone formulations after storing at 60° C. for 2 weeks.

FIG. 32 is a graph showing the viscosity of Naloxone formulations with a citrate buffer compared to an Acetate buffer after storing at 60° C. for 2 weeks.

FIG. 33 is a graph showing an assay of Naloxone formulations after storing at 60° C. for 2 weeks in citrate buffer and acetate buffer at varying doses of Naloxone.

FIG. 34 is a graph showing the presence of Naloxone degradation peaks in formulation Naloxone buffed with citrate compared to acetate buffer at various concentrations of each buffer.

FIG. 35 is a graph showing the plasma concentrations of Naloxone after a single dose of 0.27 mg/kg administered amongst three groups of male beagle dogs.

FIG. 36 is a graph showing the plasma concentrations of Naloxone after a single dose of 0.27 mg/kg administered amongst three groups of female beagle dogs.

### DETAILED DESCRIPTION

The present inventors have determined that formulations comprising a low concentration of Naloxone (<4 mg/mL) in water at pH 3-4 for injections do not meet the threshold requirements to prevent longer periods of opioid toxicity. To solve this problem, the inventors have discovered that formulations comprising high concentrations of Naloxone (for example, >8 mg/mL) are suitable to prevent renarcotization. Described herein are formulations with specific pH modifiers, buffer agents, preservatives, and tonicity modifying agents, designed to keep high doses of naloxone stable as an aqueous pharmaceutical composition.

### Definitions

As referred to herein, the term “opioid” is intended to refer to substances that act on opioid receptors to produce morphine-like effects. As a non-limiting example, some common opioids include Fentanyl, Morphine, Codeine, Tramadol, Opium, Morphine Sulfate, Levorphanol, Oxycodone, Buprenophine, Demerol, Meperidine, Carfentanil, Extended-release morphine, Methadone, Heroin, Hydromorphone, Opana, oxymorphone, Hydrocodone/Acetaminophen, Nucynta, Dextromethorphan, Dextropropoxyphene, and loperamide.

As referred to herein, the term “ultra-potent” is used to refer to compounds having a greater ability to act on opioid receptors to produce morphine-like effects compared to morphine.

As referred to herein, the term “natural opioid” refers to natural (or occurring in nature) opioid analgesics, as a non-limiting example, morphine, codeine, and thebaine.

As referred to herein, the term “synthetic opioid” refers to substances that are synthesized in a controlled setting and that act on the same targets in the brain as natural opioids.

As referred to herein, the term “poison” or “poisoning” is intended to refer to a substance that causes injury, illness, or death, particularly by acting upon the opioid receptors of a subject, and depressing the central nervous system and respiratory system, the depression manifesting as lethargy or coma, decreased respiratory rate, miosis, bradypnea, and possibly apnea (1-3) or death.

As referred to herein, the term “overdose” refers to when a subject consumes or is exposed to more than a medically recommended or physiologically tolerable amount of an opioid.

As referred to herein, the term “aqueous” means containing water.

As referred to herein, the phrase “pharmaceutically acceptable salt” refers to both pharmaceutically acceptable acid and base addition salts and solvates. Such pharmaceutically acceptable salts include salts of acids such as hydrochloric, phosphoric, hydrobromic, sulfuric, sulfinic, formic, toluenesulfonic, methanesulfonic, nitric, benzoic, citric, tartaric, maleic, hydroiodic, alkanoic such as acetic, HOOC—(CH₂)ₙ—COOH where n is 0-4, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

As referred to herein, the term “parenteral” means administered or occurring elsewhere in the body than the mouth and alimentary canal.

As referred to herein, the term “pH” refers to the acidity or alkalinity of a solution on a logarithmic scale, such that pH is equal to the −log₁₀ [H⁺], wherein [H⁺] is the concentration of hydrogen ions expressed in moles per liter.

As referred to herein, the term “pharmaceutical composition” means one or more compounds of the disclosure may be present in association with one or more pharmaceutically acceptable carriers, diluents or excipients, and, if desired, other active ingredients.

Aqueous suspensions contain the active materials in admixture with excipients suitable for the manufacture of aqueous suspensions. Such excipients can be suspending agents, for example sodium carboxymethylcellulose, methylcellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate.

Oily suspensions can be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. These compositions may be preserved by the addition of an anti-oxidant such as ascorbic acid.

In some embodiments, the pharmaceutically acceptable carrier, diluent, or excipient is not water. In other embodiments, the water comprises less than 50% of the composition. In some embodiments, compositions comprising less than 50% water have at least 1%, 2%, 3%, 4%, or 5% water. In other embodiments, the water content is present in the composition in a trace amount.

In some embodiments, the pharmaceutically acceptable carrier, diluent, or excipient is not alcohol. In other embodiments, the alcohol comprises less than 50% of the composition. In some embodiments, compositions comprising less than 50% alcohol have at least 1%, 2%, 3%, 4%, or 5% alcohol. In other embodiments, the alcohol content is present in the composition in a trace amount.

The compositions administered to a patient can be in the form of pharmaceutical compositions described above. These compositions can be sterilized by conventional sterilization techniques or may be sterile filtered. Aqueous solutions can be packaged for use as is, or lyophilized, the lyophilized preparation being combined with a sterile aqueous carrier prior to administration. The pH of the compound preparations typically will be between 1 and 11, more preferably from 2 to 9 and most preferably from 2 to 4. It will be understood that use of certain of the excipients, carriers, or stabilizers will result in the formation of pharmaceutical salts.

As referred to herein, the term “preservatives” means any compound intended to stabilize and further the shelf life of the formulation, and may include for example ethyl, or n-propyl p-hydroxybenzoate, sodium benzonate, benzalkonium chloride, phenoxyethanol, benzoic acid, acetic acid, citric acid, sorbic acid, tartaric acid, butylated hydroxytoluene, potassium sorbate, methylparaben, ethylparaben, propylparaben, butylparaben, isobutylparaben, parahydroxybenzoates or esters thereof, isothiazolinones, calcium phosphate, calcium propionate, sodium chloride, sucrose, potassium metabisulfite, and sodium nitrate. Those skilled in the art will recognize a wide variety of preservatives may be employed and that some preservatives may also act as a buffering agent, tonicity modifier, or pH modifier while simultaneously serving as a preservative.

As referred to herein, the term “buffering agents” refer to solutions that can resist changes in pH and include, for example, glycine-sodium hydroxide, monobasic or dibasic sodium phosphate, sodium carbonate, bicarbonate, potassium chloride, potassium phosphate, sodium chloride, acetic acid, acetate, boric acid, hydroxycarboxylic acid compounds such as sodium citrate, trisodium citrate dihydride, citric acid, citric acid monohydrate, glycolic acid, hydroxypropionic acids (e.g., lactic acid), hydroxybutyric acids (beta-hydroxybutyric acid), salicylic acid, and ricinoleic acid.

As referred to herein, the term “tonicity modifier” refers to compounds capable of modifying the tonicity of a solution and include, for example, dextrose, glycerin, mannitol, potassium chloride, sodium chloride, sucrose, trehalose, maltose, sorbitol, or the like. Those skilled in the art will recognize a wide variety of tonicity modifiers may be employed and that some tonicity modifiers may also act as buffering agents, or pH modifiers while simultaneously serving as a tonicity modifier.

As referred to herein, the term “pH modifier” refers to a component of an aqueous solution that acidifies or alkalizes the overall solution. Some non-limiting examples of pH modifiers include adipic acid, tartaric acid, acetic acid, ammonia, ammonium hydroxide, anhydrous citric acid, choline chloride, sodium bicarbonate, sodium hydrogen carbonate, tri-potassium citrate monohydrate, potassium hydroxide, hydrochloric acid, sodium hydroxide, monobasic sodium phosphate anhydrous, dibasic sodium phosphate dodecahydrate, phosphoric acid, glacial acetic acid, sulfuric acid, and the like. Those skilled in the art will recognize that a wide variety of pH modifiers may be employed and that some pH modifiers may also act as buffering agents, or tonicity modifiers while simultaneously serving as a pH modifier.

As referred to herein, the term “Naloxone” refers to the compound Naloxone Hydrochloride, also known as (17-Allyl-6-deoxy-7,8-dihydro-14-hydroxy-6-oxo-17-normorphine Hydrochloride) or ((−)-17-Allyl-4, 5 α-epoxy-3,14-dihydroxymorphinan-6-one hydrochloride), and possessing the molecular formula C₁₉H₂₁NO₄·HCl. Naloxone may be present in its dihydrate form as well.

As referred to herein, the term “bioavailability” refers to the proportion of a drug or other substance which enters the circulation when introduced into a subject and so is able to have an active effect.

As referred to herein, the term “intramuscular” means situated or taking place within, or administered into, a muscle.

As referred to herein, the term “intravenous” is an injection of a medication or other substance into a vein or artery of a subject and thereby directly into the bloodstream.

As referred to herein, the term “subcutaneous” is used to refer to the fatty tissues just beneath the skin. A person of skill in the art would appreciate that a subcutaneous injection will be shallower than an intramuscular injection.

As referred to herein, the term “impurity” refers to chemical compounds confined within a formulation that differ from the expected ingredients of that formulation. In the case of Naloxone formulations, impurities often occur from species derived from degraded forms of Naloxone. Examples of such Naloxone degradation species include but are not limited to the following: noroxymorphone, 3-0-Allylnaloxone, 10-α-Hydroxynaloxone, 7,8-didehydronaloxone, 2,2′-bisnaloxone, and 10-β-Hydroxynaloxone.

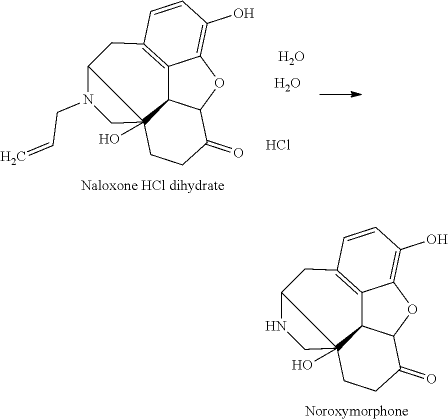

For example, Noroxymorphone is a major degradation impurity of Naloxone.

As referred to herein, the term “relative humidity” also denoted as “RH” is intended to mean the amount of water vapor present in air expressed as a percentage of the amount needed for saturation at the same temperature.

As referred to herein, the term “vial” refers to a small glass or plastic vessel or bottle, used to store formulations in the form of a liquid. The vial may be colorless or amber. Additionally, the vial may possess a screw-on cap, or may be sealed with a cork or rubber stopper, or may be crimped closed with a rubber stopper and a metal cap. The vial may be sealed with a polytetrafluoroethylene (PTFE) septum, and the PTFE septum may be pierced with a needle to access the contents of the vial. The septum may also be made of silicone entirely or may be made of silicone but laminated with PTFE. A person skilled in the art will recognize that many vials may be suitable to house a high dose Naloxone formulation.

As referred to herein, the term “dose” is understood to mean a measured quantity of a medicine, particularly a formulation of Naloxone, which is delivered as a unit; the greater the quantity delivered, the higher the dose. “Dose” may also be referred to as a therapeutic dose. Doses may be expressed in mg/ml or mg/ml (milligrams per milliliter), mg/kg (milligrams per kilogram), μg/kg (micrograms per kilogram), or any other acceptable unit of measurement. The therapeutic dosage of the Naloxone formulation can vary according to, for example, the particular use for which the treatment is made, the manner of administration, the health and condition of the patient, and the judgment of the prescribing physician. The dosage is likely to depend on such variables as the type and extent of progression of the opioid poisoning, the overall health status of the particular patient, the relative biological efficacy of the formulation selected, choice of excipients, and the rout of administration. A person skilled in the art will understand that units for dosing depend on the size of batch to be formulated as well as the size of the target the formulation is intended to be administered to.

As referred to herein, the term “photo-degradation” or “photodegradation” refers to the alteration of materials by light. Specifically, the degradation of Naloxone trough reactions with electromagnetic wavelengths found in sunlight, such as infrared radiation, visible light, and ultraviolet light.

As referred to herein, the term “about” generally means within 10%, 5%, 1%, or 0.5% of a given value or range. Alternatively, the term “about” means within an acceptable standard error of the mean when considered by one of ordinary skill in the art. Other than as provided in the operating/working examples, or unless otherwise explicitly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and claims are approximations that can vary as desired. At the very least, each numerical parameter should at least be construed in light of the number of reported significant digits and by applying ordinary rounding techniques.

As referred to herein, the term “auto injector” or “autoinjector” or “auto-injector” means a medical device for injecting oneself, or another, with a single, preloaded dose of a drug that typically consists of a spring-loaded syringe activated when the device is pushed firmly against the body. The autoinjector may also be a pen injector. In some embodiments, when using an autoinjector, the patient actuates a needle and flow of medication only when pressure is applied to an injection site. The pressure causes the actuation of a needle safety shield, which engages the needle and causes the device to inject the drug into a patient. Pen injector devices require a patient to activate a push-button, which actuates a needle into a targeted injection site. A person skilled in the art will understand that both an autoinjector and an injector pen are similar from a mechanical perspective and either may be used to deliver an aqueous pharmaceutical composition of Naloxone as further described herein.

As referred to herein, the term “testing apparatus” may refer to any diagnostic test capable of detecting the presence of opioids. As a non-limiting example, one such testing apparatus is a test strip capable of detecting fentanyl as described by Krieger et al., Int. J. Drug Policy 2018 November; 61:52-58. Other testing apparatuses are also possible such as urine tests, or rapid blood tests. A person skilled in the art recognizes that there are many ways to detect opioids and therefore, many suitable testing apparatuses.

As referred to herein, the term “product use pamphlet” is also synonymous with “package insert” or “product insert” and includes details and directions that first responders or lay persons need to know in order to properly administer, or self-administer, a therapeutic dose of Naloxone formulation required to reverse or ameliorate the effects of opioid-poisoning.

### DETAILED DESCRIPTION OF THE FIGURES

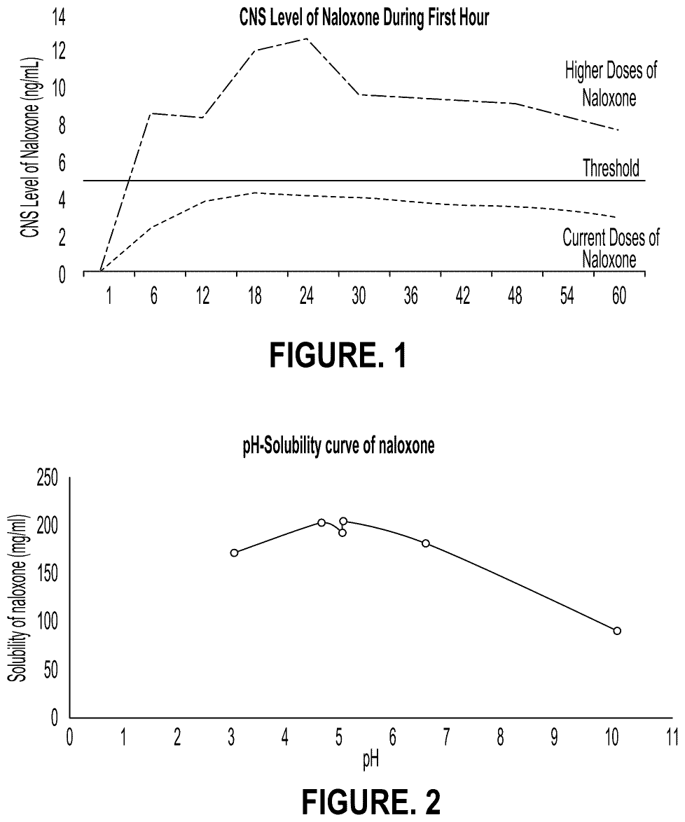

FIG. 1 is a graph showing hypothetical Naloxone concentration in the central nervous system comparing high-dose naloxone verses traditional doses of Naloxone. Naloxone, the only medication approved by FDA for the treatment of opioid overdose, is identified by the World Health Organization (WHO) as an effective antidote to opioid overdose, able to completely reverse the effects of an overdose if administered in a timely manner. Although an exact dose has not been identified, available literature advocates the need for multiple doses of naloxone to counter overdose of synthetic opioids. The use of increased doses of naloxone to achieve higher thresholds of effect for synthetic opioids relative to current and higher doses is illustrated in FIG. 1.

FIG. 2 is a graph showing the solubility of Naloxone relative to increasing pH. Naloxone HCl showed maximum solubility of 204.29 mg/ml in citric acid/sodium citrate buffer at pH 5.0 and lowest of 90.54 mg/ml in glycine-sodium hydroxide buffer at pH 10.0. A decrease in pH of the filtrate solution was observed in buffers samples at 6.5 and 10.0. This suggest that naloxone hydrochloride solution needs buffer strength greater than 10 mM formulations prepared in buffers greater than 5.0. A light-yellow color of naloxone hydrochloride solution was observed in saturation solubility samples with the intensity of color being stronger at higher pH value.

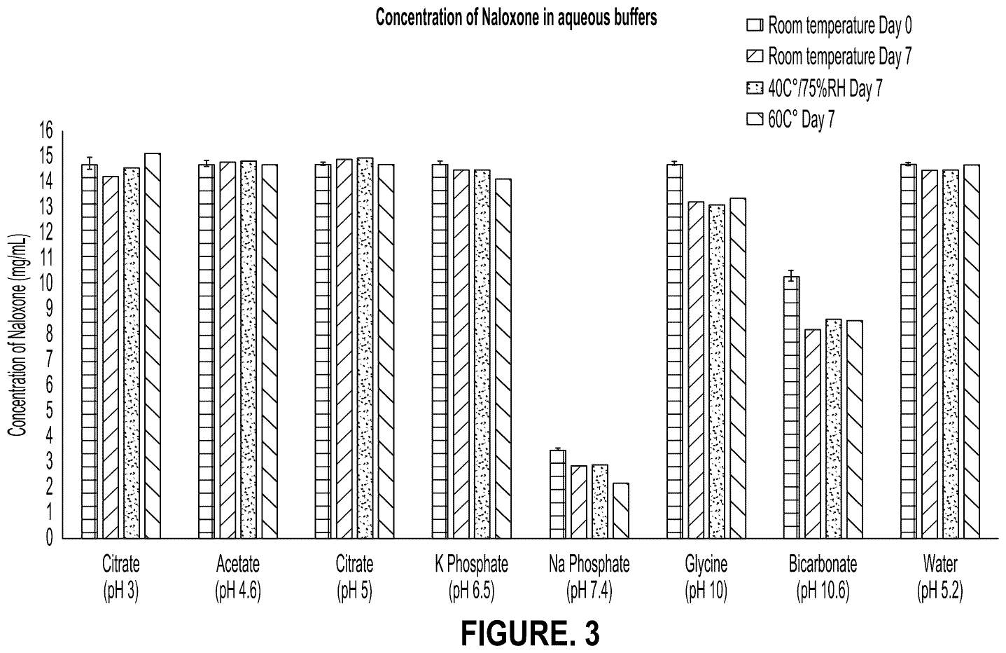

FIG. 3 is a graph that depicts the concentration of Naloxone in various buffers at t=0 and t=7. The t=7 represents samples stored at either 40° C. or 60° C. for up to 7 days. FIG. 3 presents the drug concentrations found to be solubilized in various pH buffers; the maximum possible solubility of each sample is 15 mg/ml because excess drug was not added to the buffer systems. The 15 mg/ml was chosen for pH stability studies and a final formulation of Naloxone is planned to be at 10 mg/ml strength. The maximum possible solubility of drug substance at various buffers will be performed in a “pH solubility” study. Naloxone hydrochloride did not dissolve efficiently in buffer systems at pH 7.4 (sodium phosphate monobasic/dibasic) and pH 10.6 (carbonate-bicarbonate). Naloxone precipitated in solution maintained at pH 7.4-10.6. The glycine-sodium hydroxide buffer system fared slightly better, however not as well as the buffers that maintained pH at 3.0-6.5. It is likely that buffer strength higher than 10 mM is required for pH 7.0-11 buffer systems. Naloxone hydrochloride has three pKas: pKa1=7.84; pKa2=10.07; pKa3=13.58. Based on the pKa values, solubility of Naloxone is expected to be higher at lower pH conditions.

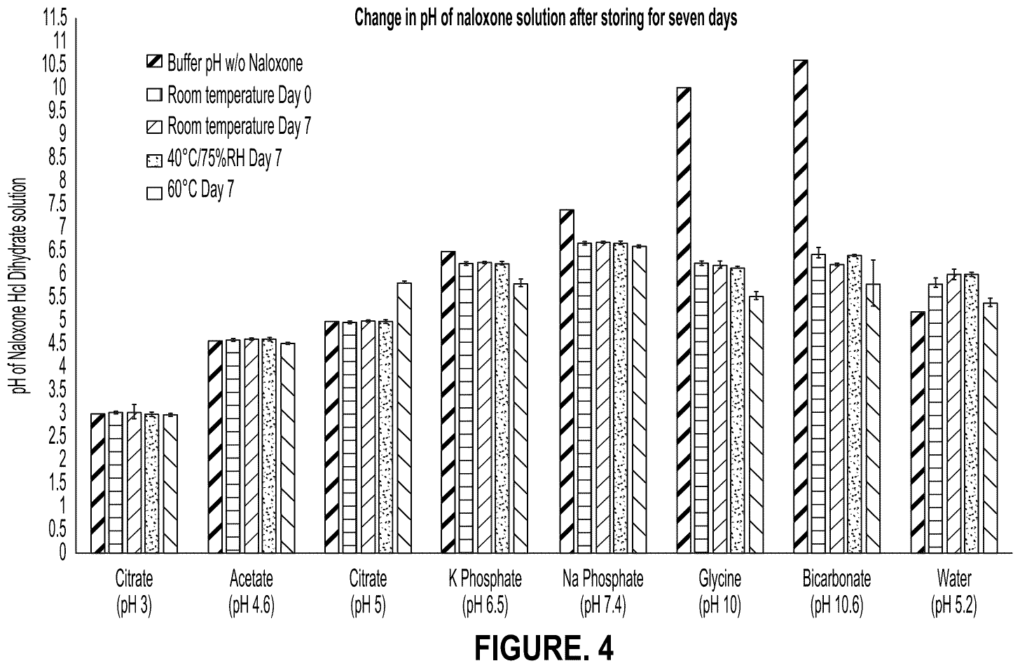

FIG. 4 is a graph that depicts the change in pH of Naloxone in various buffers t=0 and t=7. The t=7 represents samples stored at either 40° C. or 60° C. for up to 7 days. FIG. 4 follows the change in pH for the buffered naloxone samples as a function of time and temperature of storage. It appears that higher pH instability and higher temperature degradations are concerns for Naloxone formulations. Optimum buffering of Naloxone appears to occur at pH 3-4.6; at the molarity tested.

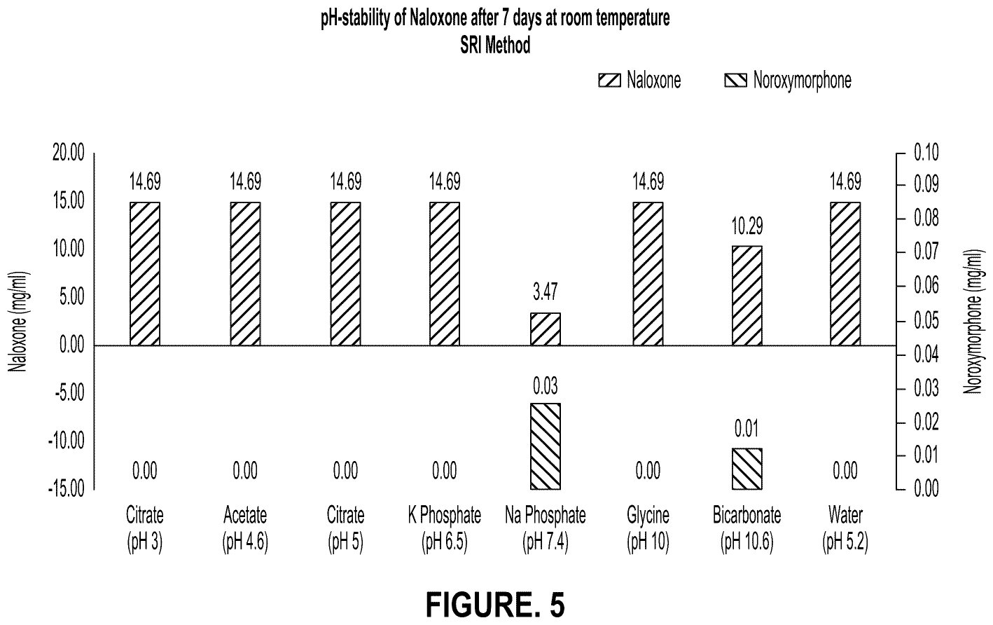

FIG. 5 is a graph that depicts pH-stability of Naloxone after 7 days at room temperature by showing Naloxone and Noroxymorphone concentrations in various buffers assayed by HPLC. Temperature and duration of sample storage are at ambient temperature for 7 days. Greater amounts of Noroxymorphone were detected at pH 7.4 in a sodium phosphate buffer and a pH of 10 in a glycine buffer than at pHs lower than 6.5. This suggests that Naloxone is less stable at high pHs, that is greater than 7.4.

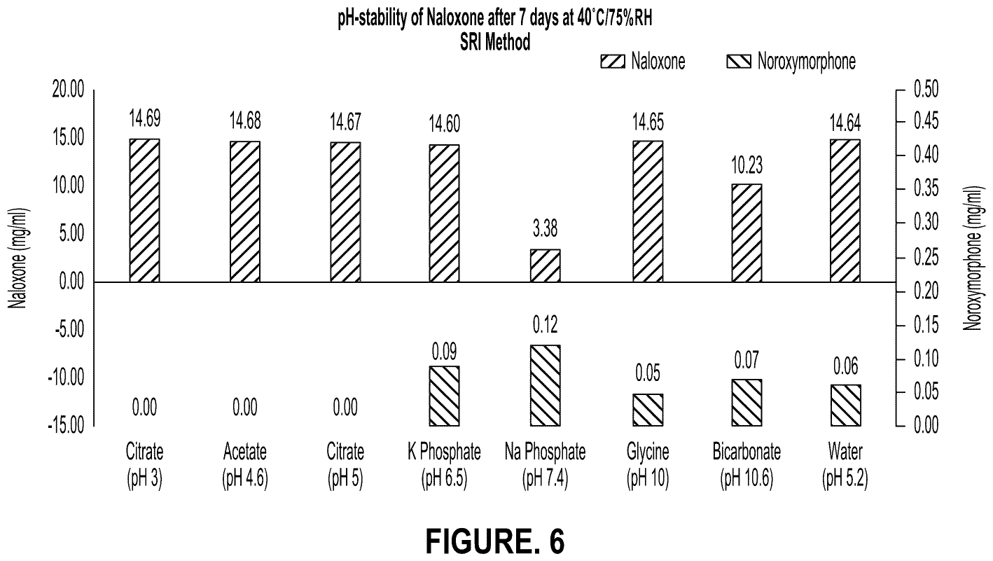

FIG. 6 is a graph that depicts pH-stability of Naloxone after 7 days at 40° C. and 75% relative humidity by showing Naloxone and Noroxymorphone concentrations in various buffers assayed by HPLC. Temperature and duration of sample storage are at 40° C. and 75% relative humidity for 7 days. FIG. 6 follows up on the conclusions drawn from the experiment conducted that underlies FIG. 5. Raising the temperature incudes greater formation of Noroxymorphone, and this effect is more pronounced in formulations with a higher pH.

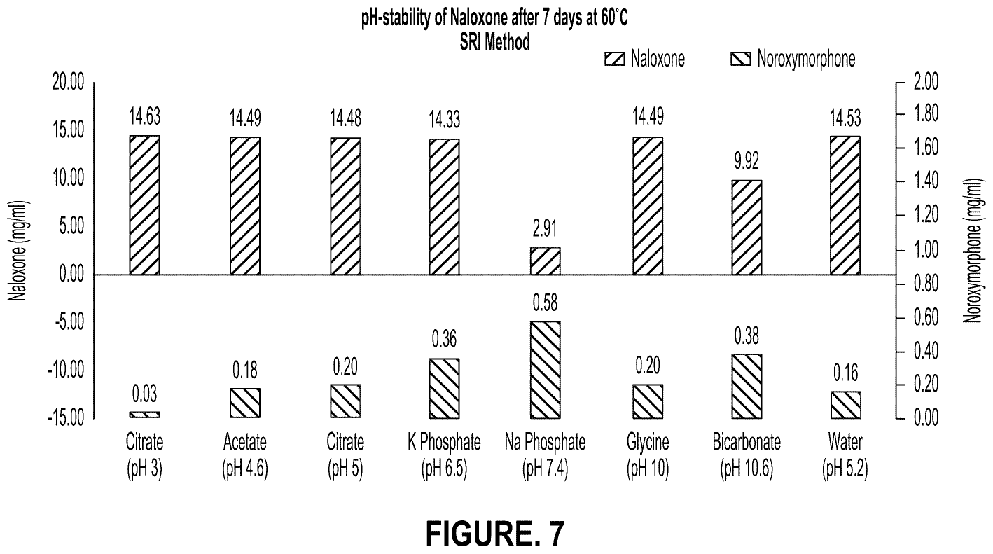

FIG. 7 is a graph that depicts pH-stability of Naloxone after 7 days at 60° C. by showing Naloxone and Noroxymorphone concentrations in various buffers assayed by HPLC. Temperature and duration of sample storage are at 60° C. and humidity was not maintained for 7 days. At even higher temperatures, Noroxymorphone was detected in all formulations across all respective pHs. Notably, the formulation with a citrate buffer at pH 3 showed significantly less Noroxymorphone formation than all of the other formulations tested.

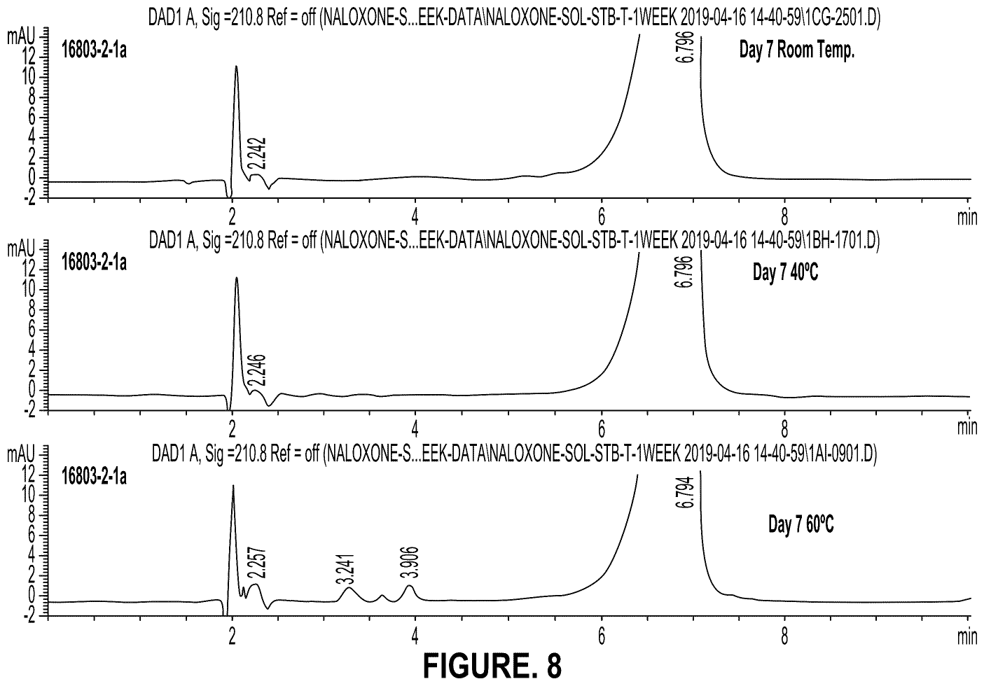

FIG. 8 is an HPLC chromatogram of naloxone degradation in a citrate buffer with pH 3.0 after storage for 7 days.

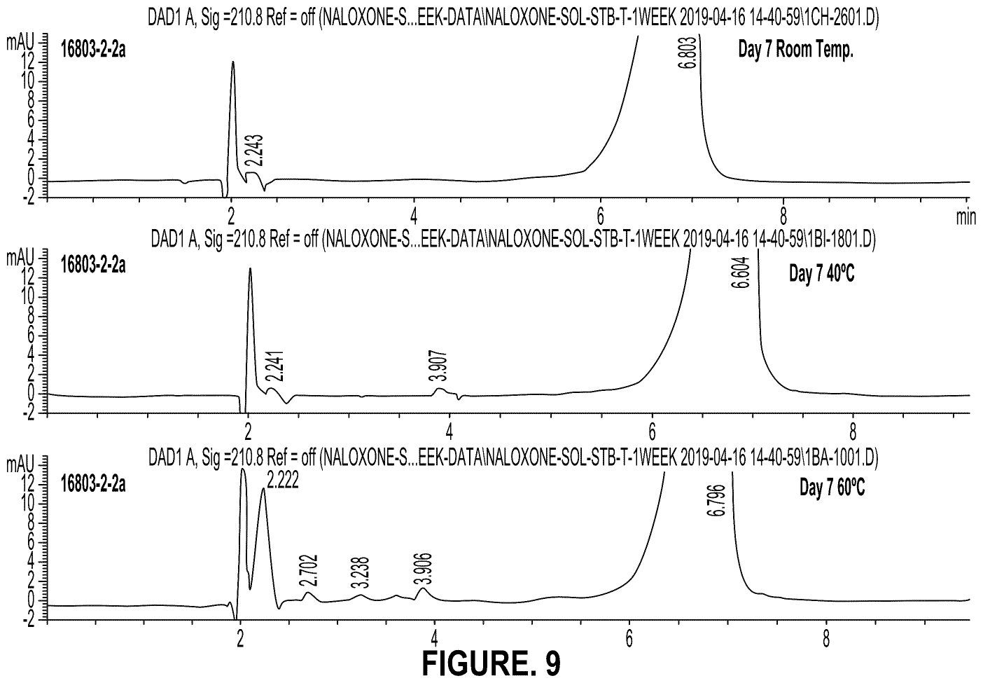

FIG. 9 is an HPLC chromatogram of naloxone degradation in a citrate buffer with pH 4.6 after storage for 7 days.

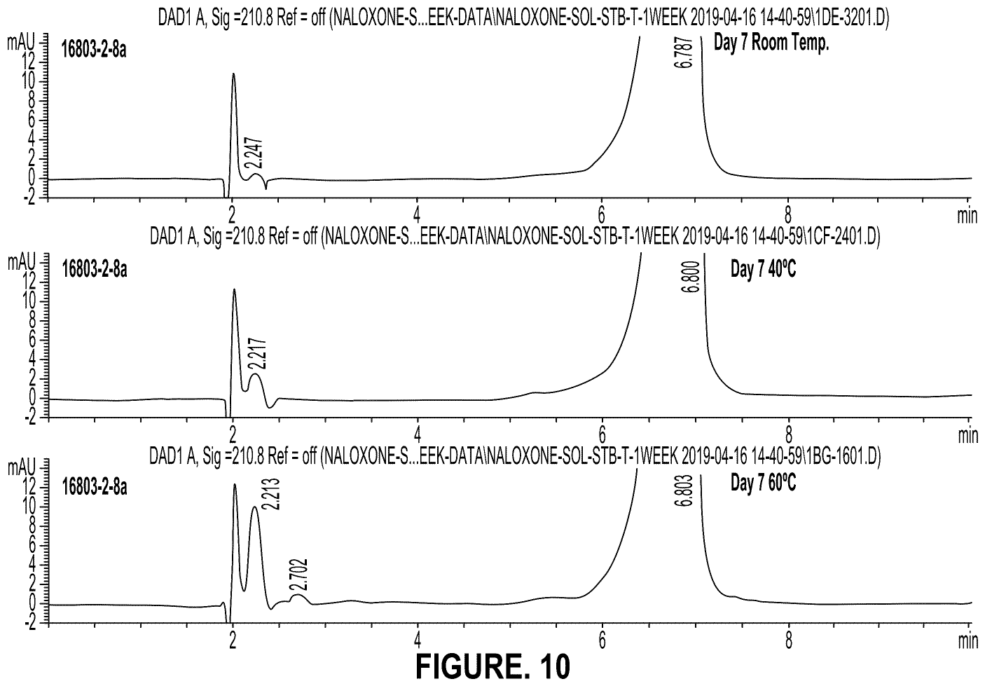

FIG. 10 is an HPLC chromatogram of naloxone degradation in a citrate buffer with pH 5.2 after storage for 7 days.

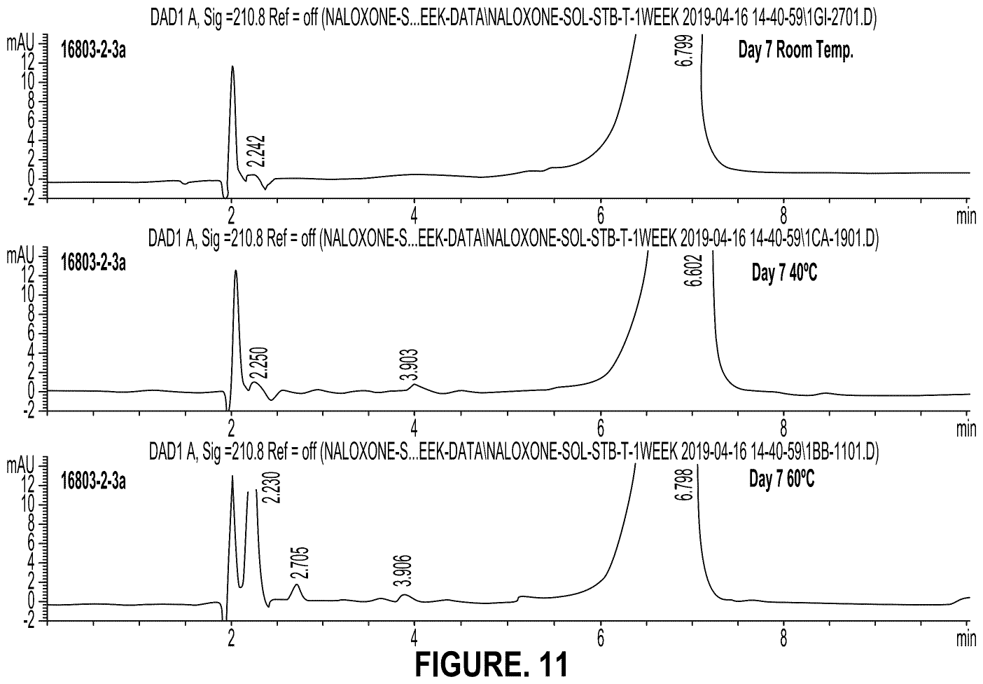

FIG. 11 is an HPLC chromatogram of naloxone degradation in a citrate buffer with pH 5.0 after storage for 7 days.

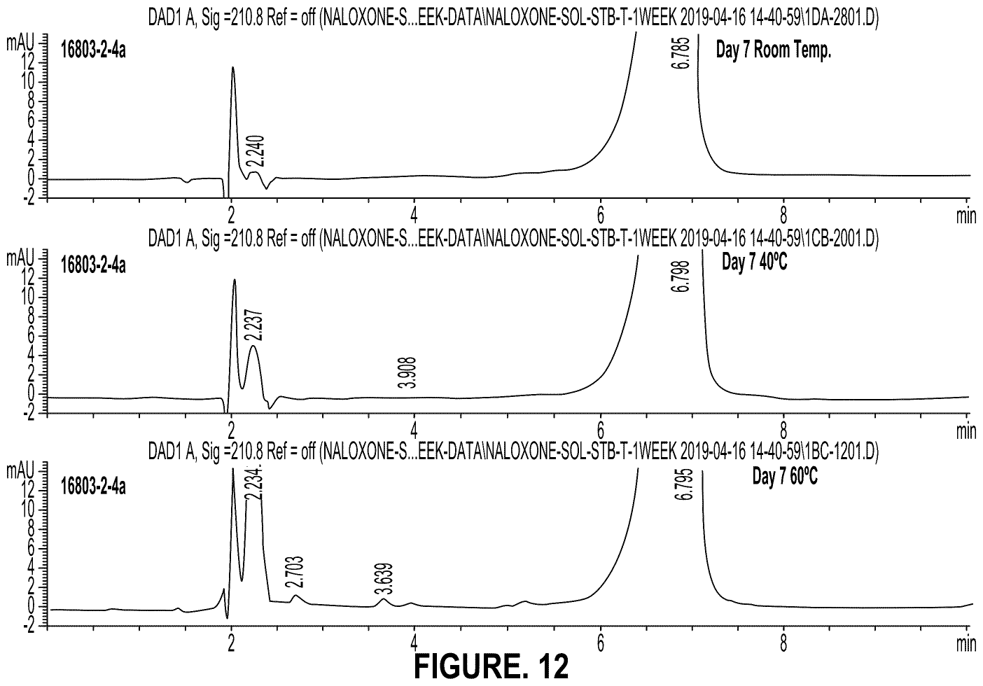

FIG. 12 is an HPLC chromatogram of naloxone degradation in a potassium phosphate buffer with pH 6.5 after storage for 7 days.

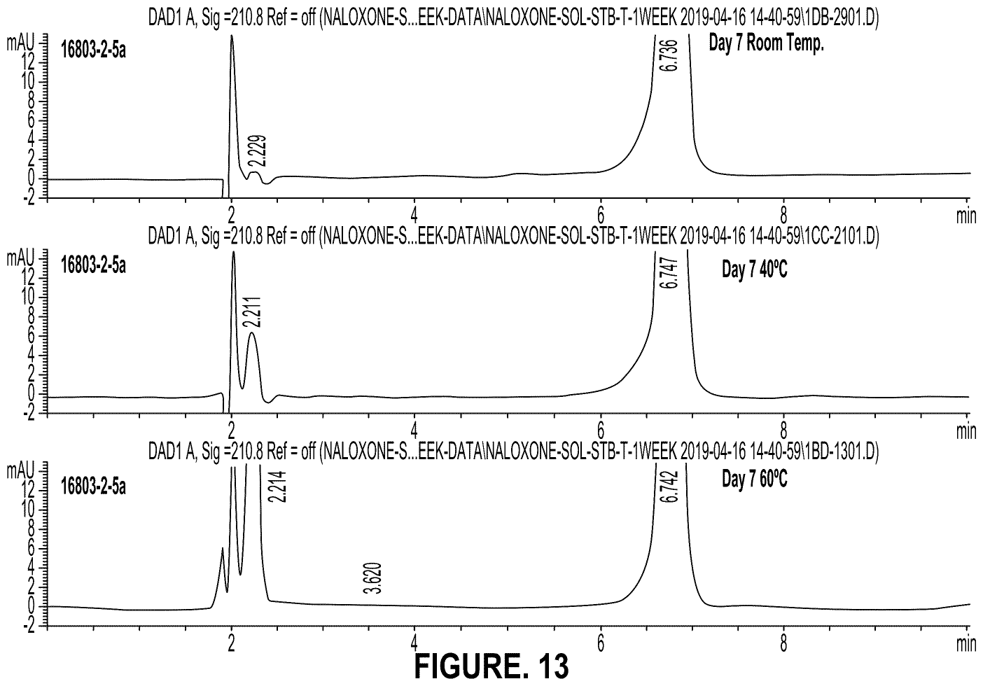

FIG. 13 is an HPLC chromatogram of naloxone degradation in a sodium phosphate buffer with pH 7.4 after storage for 7 days.

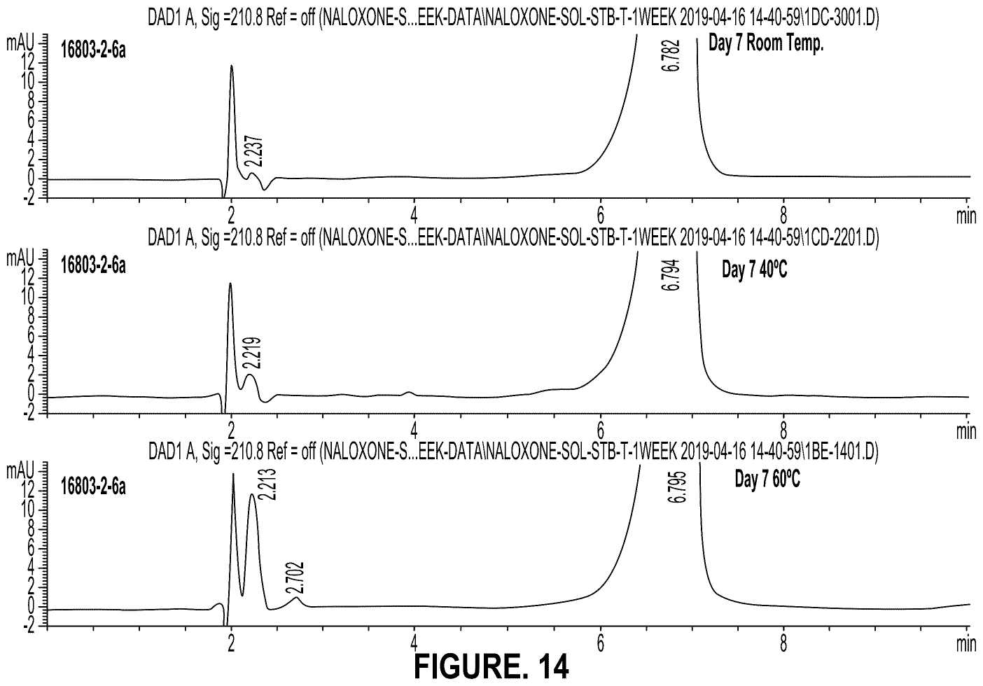

FIG. 14 is an HPLC chromatogram of naloxone degradation in a glycine-sodium hydroxide buffer with pH 10.0 after storage for 7 days.

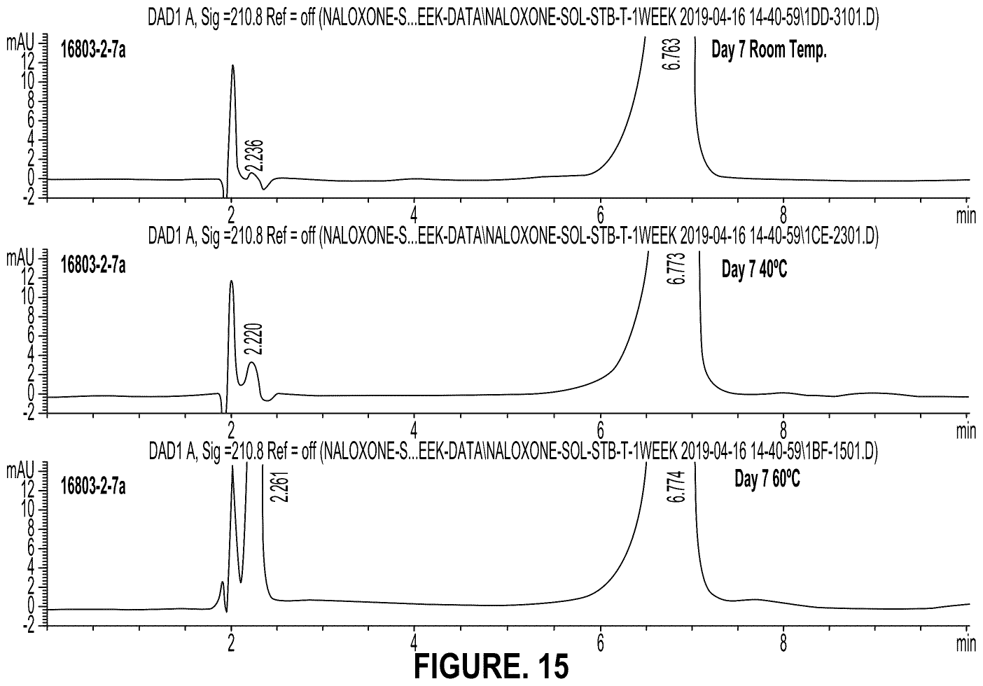

FIG. 15 is an HPLC chromatogram of naloxone degradation in a sodium carbonate-bicarbonate buffer with pH 10.6 after storage for 7 days.

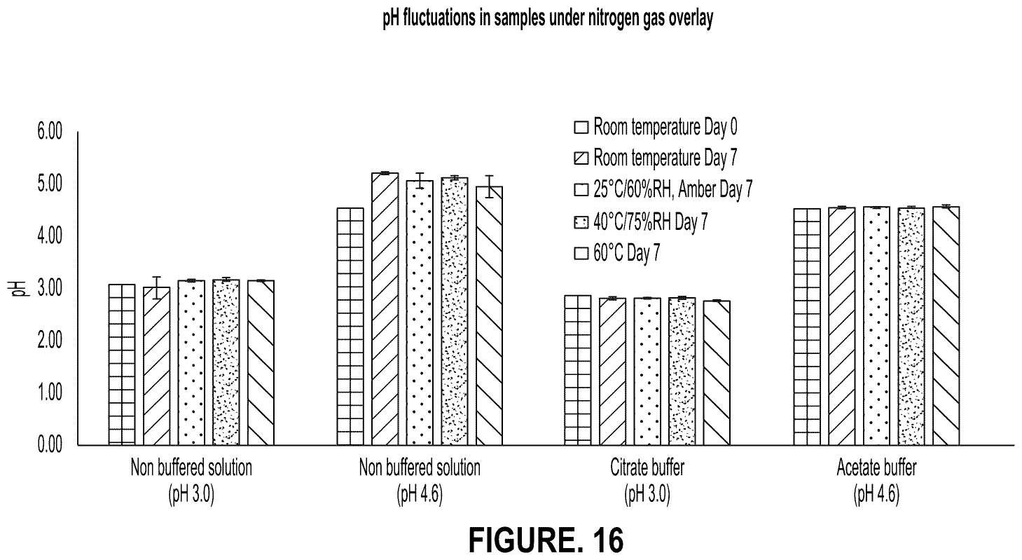

FIG. 16 is a graph showing pH fluctuations in samples under nitrogen gas overlay, specifically change in pH of Naloxone solutions in water and buffers at pH 3.0 and 4.6 on Day 0 and Day 7. The Day 7 represents samples stored at either room temperature, 40° C. and 75% relative humidity or 60° C. or 25° C. and 60% relative humidity (in amber vials) for up to 7 days. The pH fluctuations in naloxone HCl solution samples on Day 0 and Day 7 at different conditions is presented in FIG. 16. The pH of naloxone hydrochloride samples prepared without buffers at pH 3.0 exhibited no change in pH after storing for seven days at room temperature, at 40° C./75% RH, at 60° C., and at 25° C./60% RH (amber vials), and the pH of the samples prepared without buffers at pH 4.6 increased upon storing for seven days. In contrast, naloxone samples prepared in 10 mM strength citrate and acetate buffers at pH 3.0 and 4.6, respectively, exhibited no change in pH even after storing for seven days at room temperature, at 40° C./75% RH, at 60° C., and at 25° C./60% RH (amber vials). Based on these results, nitrogen gas overlay and use of buffers (citrate and acetate) are proposed as useful additions to the final formulation.

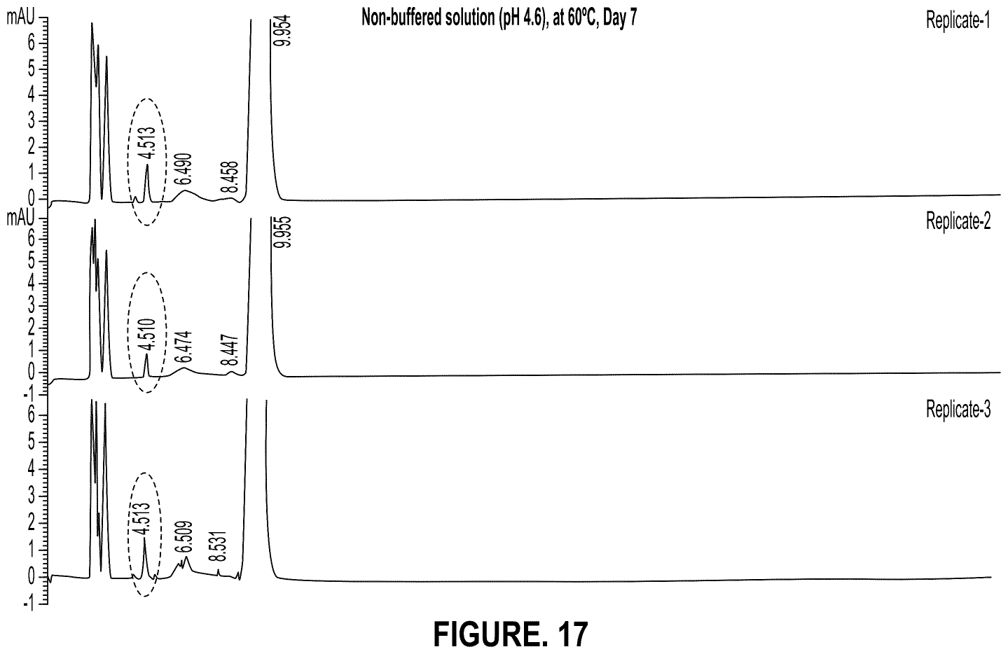

FIG. 17 is an HPLC chromatogram of naloxone degradation in a non-buffered solution at pH 4.6 at 60° C. after storage for 7 days.

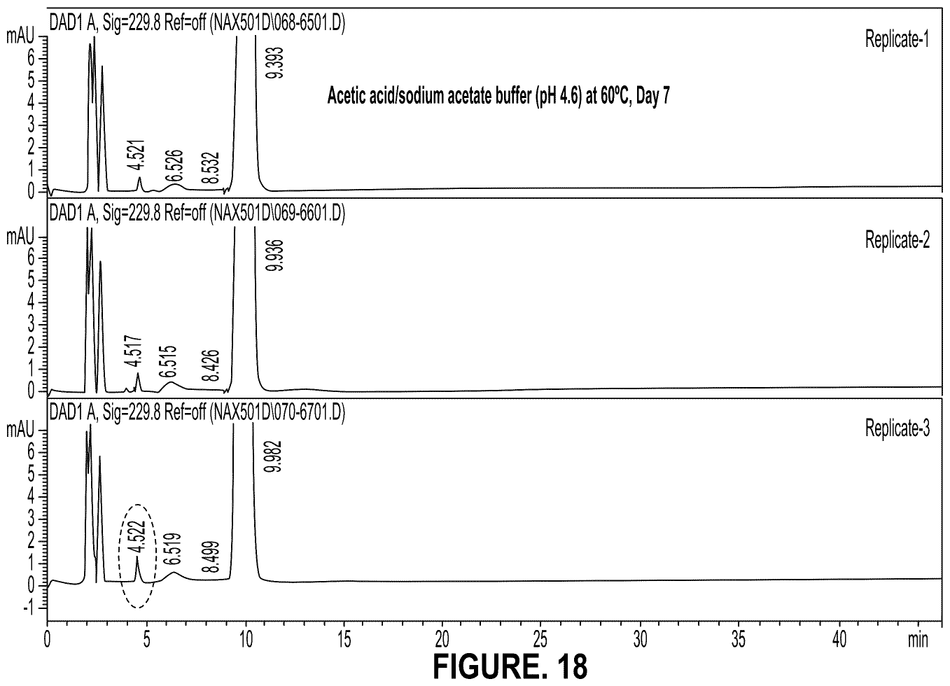

FIG. 18 is an HPLC chromatogram of naloxone degradation in a acetic acid/sodium acetate buffer solution at pH 4.6 at 60° C. after storage for 7 days.

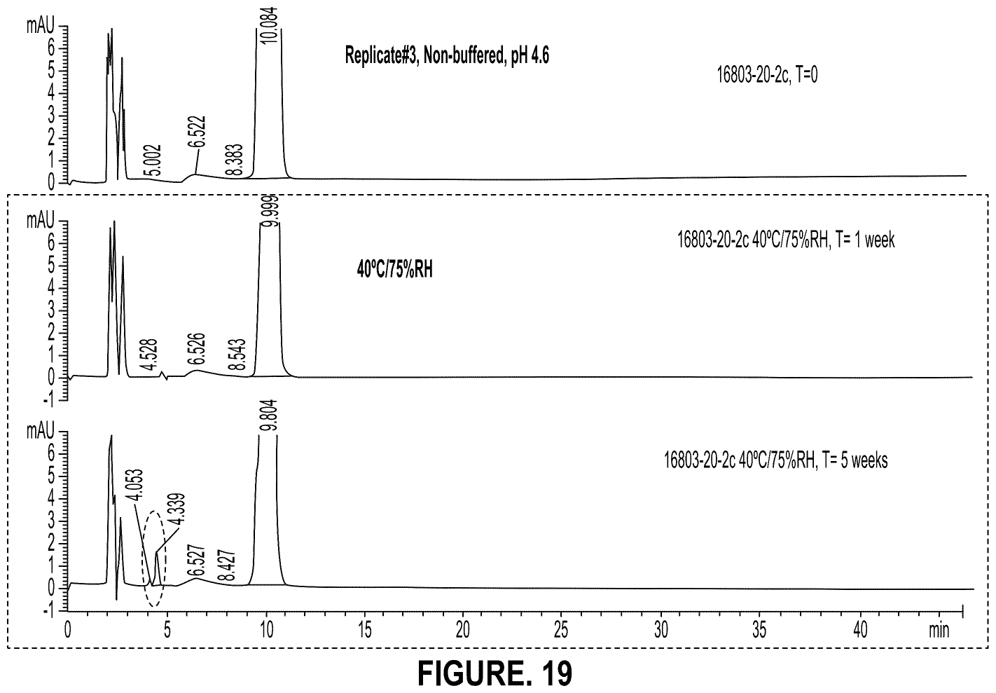

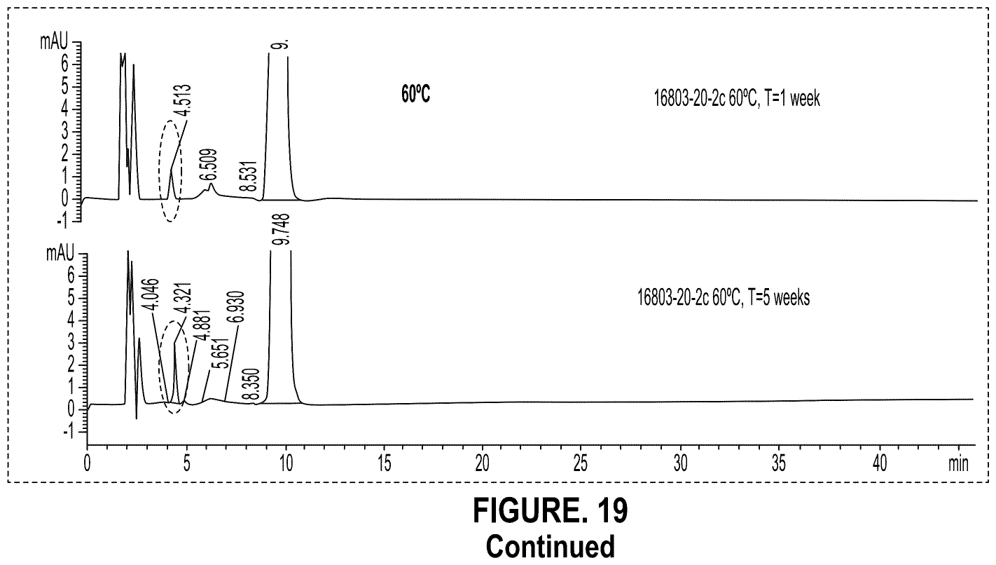

FIG. 19 is a comparison of HPLC chromatograms of naloxone degradation in a non-buffered solution at pH 4.6 at room temperature, 40° C. and 75% relative humidity, and 60° C. after storage for 5 weeks.

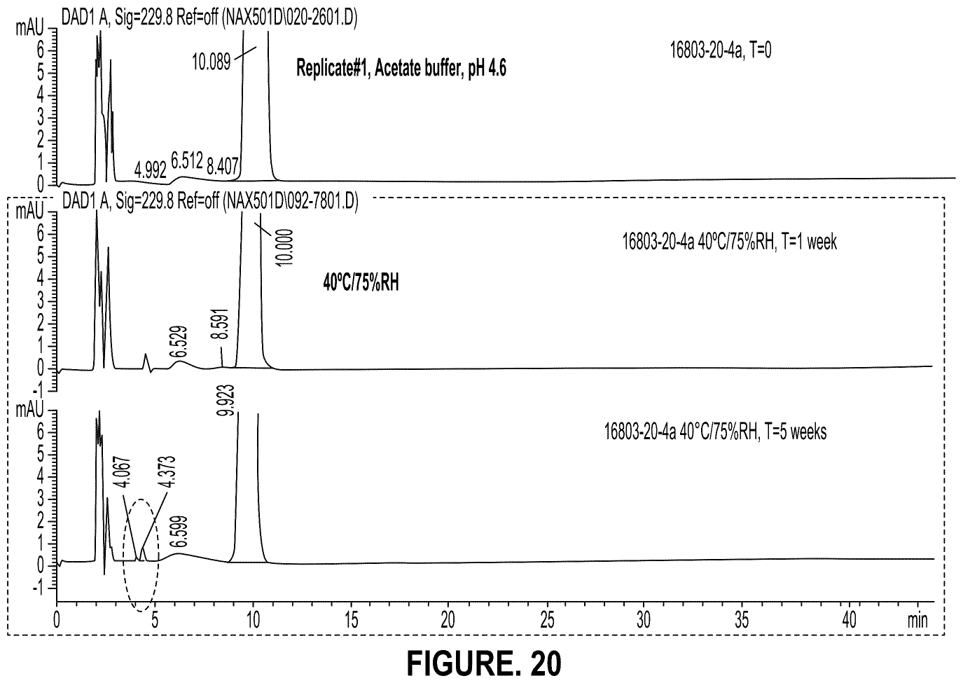

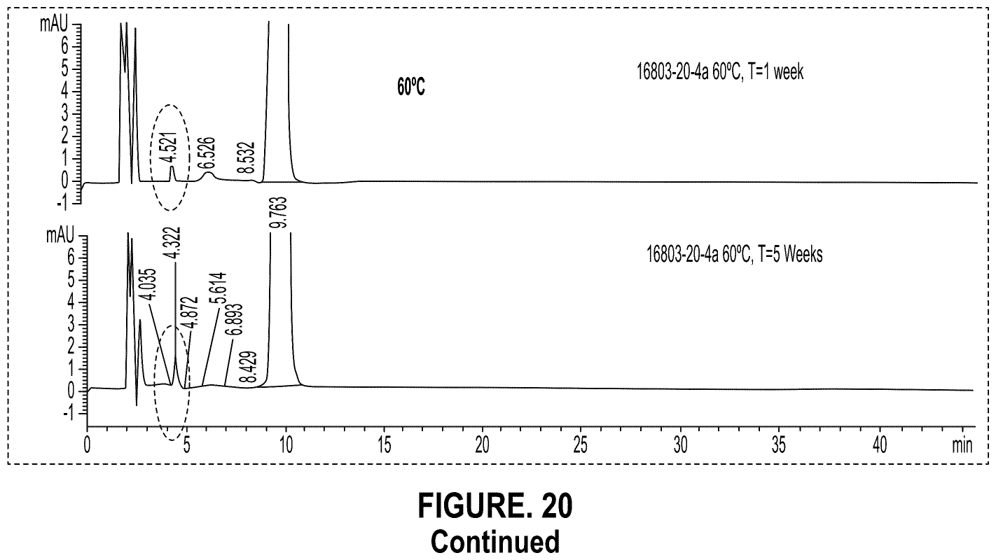

FIG. 20 is a comparison of HPLC chromatograms of naloxone degradation in an acetate-buffered solution at pH 4.6 at room temperature, 40° C. and 75% relative humidity, and 60° C. after storage for 5 weeks.

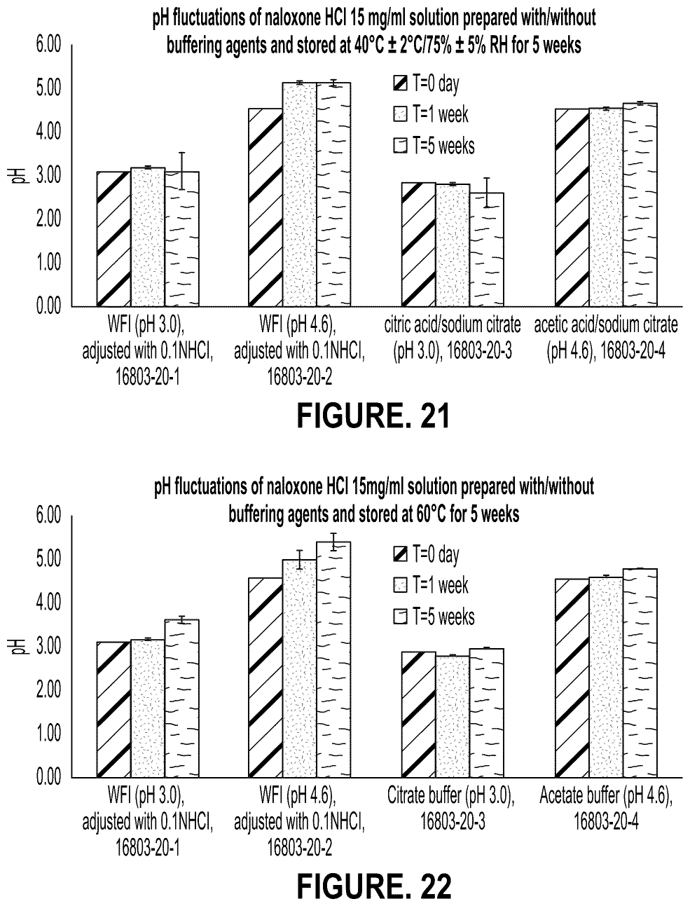

FIG. 21 is a graph showing pH fluctuations of Naloxone with different buffing agents and stored at 40° C. and 75% relative humidity for 5 weeks. Notably, the pH fluctuations are comparable in the citric acid/sodium citrate buffer at a pH of 3 to the acetic acid/sodium citrate buffer at a pH of 4.6. Both buffer systems are superior to controls with water adjusted to a pH of 3 and 4.6.

FIG. 22 is a graph showing pH fluctuations of Naloxone with different buffing agents and stored at 60° C. for 5 weeks. Notably, the pH fluctuations are comparable in the citric acid/sodium citrate buffer at a pH of 3 to the acetic acid/sodium citrate buffer at a pH of 4.6. Both buffer systems are superior to controls with water adjusted to a pH of 3 and 4.6.

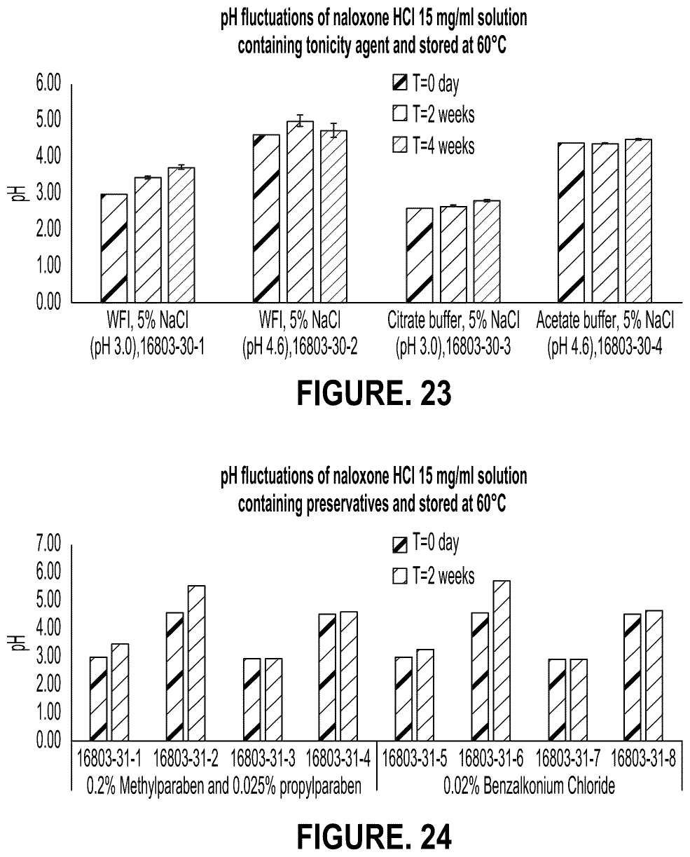

FIG. 23 is a graph showing pH fluctuations of Naloxone at a dose of 15 mg/mL containing sodium chloride as a tonicity modifying agent stored at 60° C. for 2 and 4 weeks. Both buffer systems are superior to controls with water adjusted to a pH of 3 and 4.6 even with a tonicity modifying agent.

FIG. 24 is a graph showing pH fluctuations of Naloxone at a dose of 15 mg/mL containing benzalkonium chloride, methylparaben, and propylparaben as preservatives stored at 60° C. for 2 weeks. Notably, the pH stayed most consistent in formulation 16803-31-3 (methylparaben, propylparaben, citric acid, trisodium citrate dihydrate, water, pH 3) and 16803-31-7 (citric acid, trisodium citrate dihydride, benzalkonium chloride, water, pH 4.6).

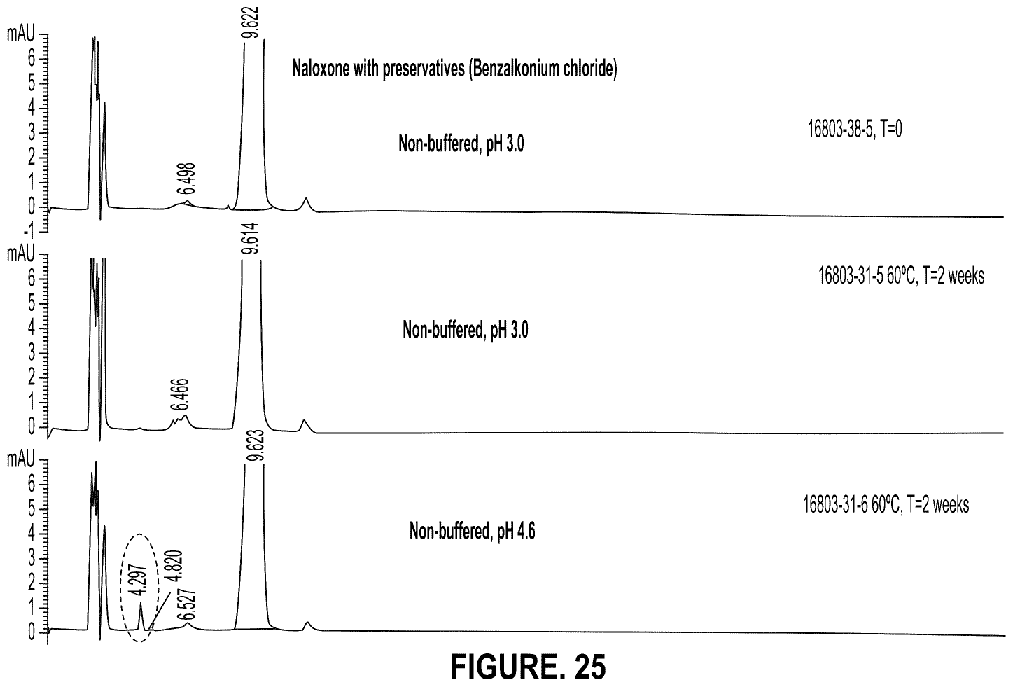

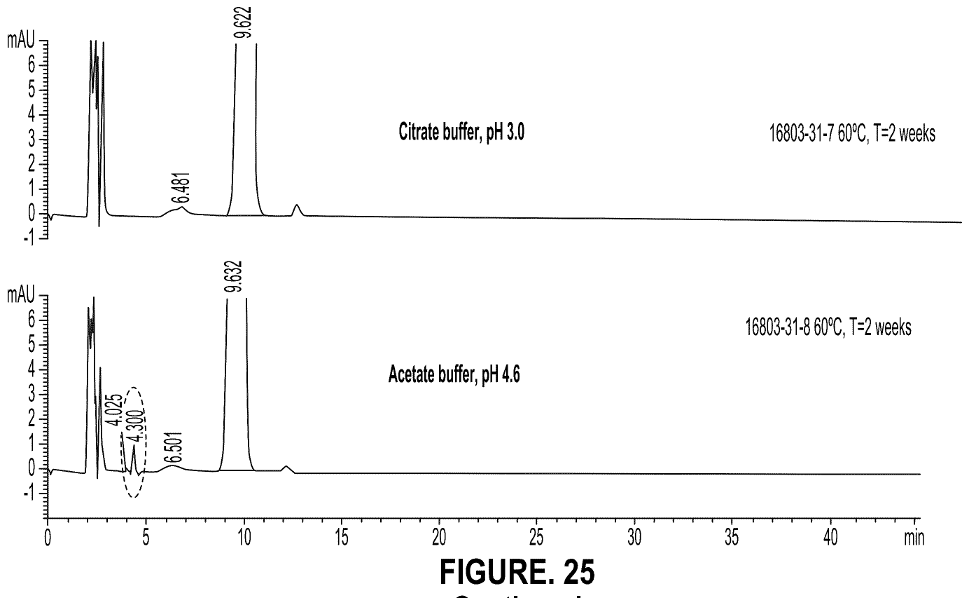

FIG. 25 is a comparison of HPLC chromatograms of naloxone degradation with benzalkonium chloride as a preservative coupled with various buffering agents at pHs of 3.0 and 4.6 at 60° C. after storage for 2 weeks.

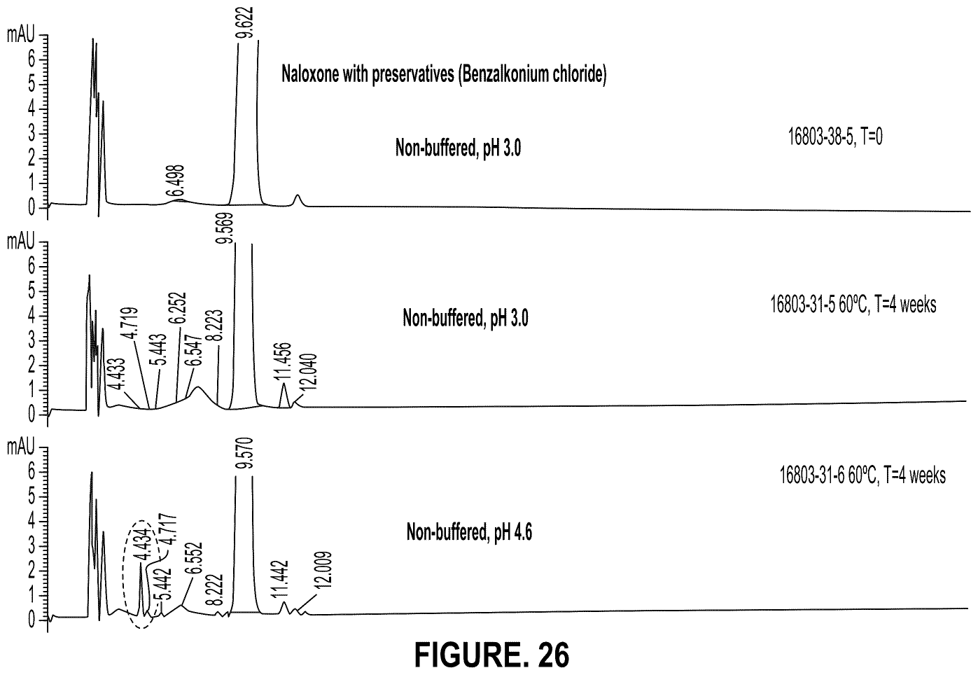

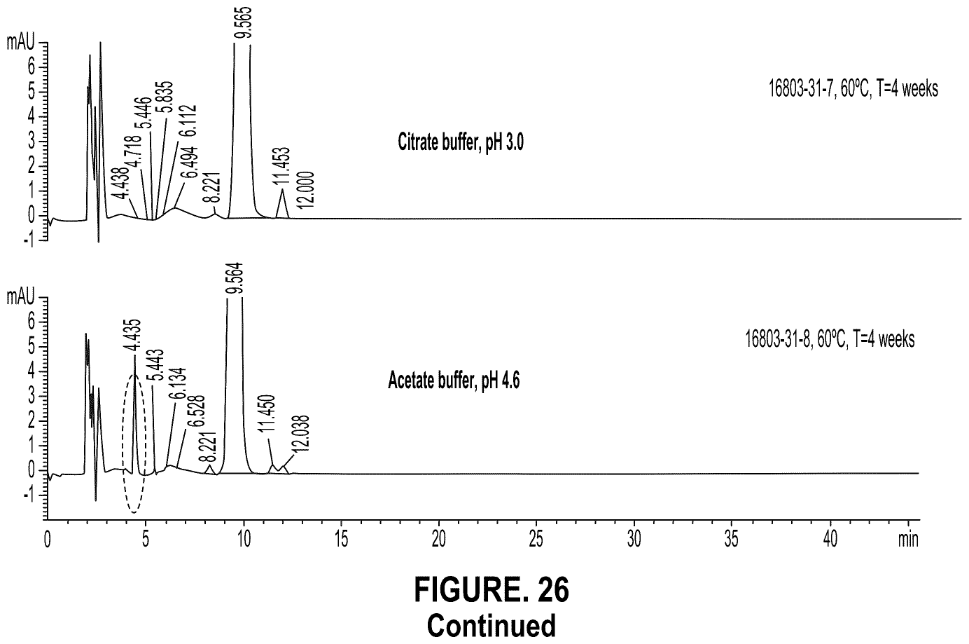

FIG. 26 is a comparison of HPLC chromatograms of naloxone degradation with benzalkonium chloride as a preservative coupled with various buffering agents at pHs of 3.0 and 4.6 at 60° C. after storage for 4 weeks.

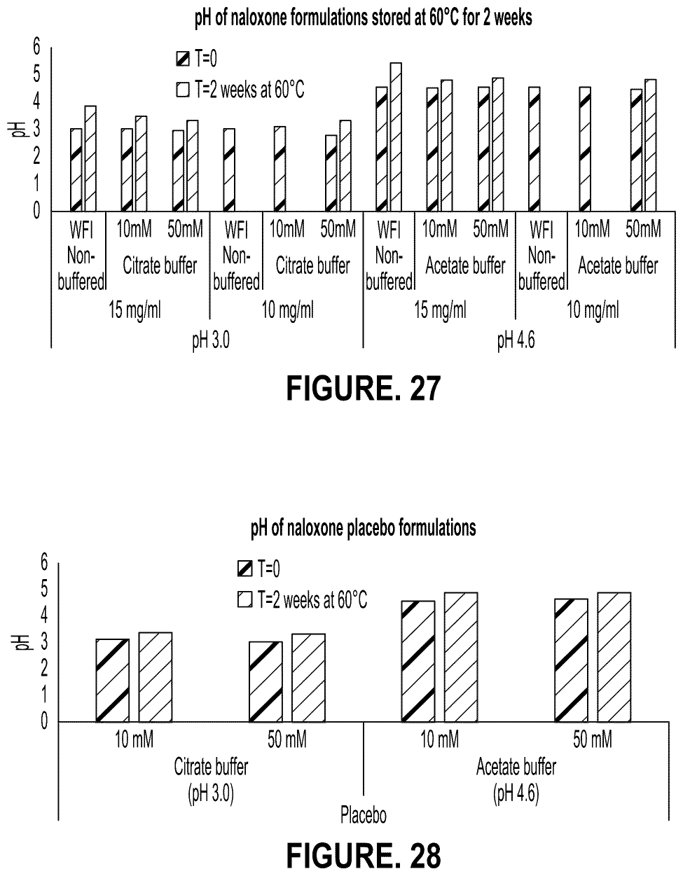

FIG. 27 is a graph showing the fluctuations of pH of Naloxone formulations stored at 60° C. for 2 weeks in various buffer systems with changing Naloxone dosing. The pH of the prepared naloxone formulations was measured using pH meter (Model 340, Corning). Based on the pH results of formulations, buffer strengths 10 mM and 50 mM for citrate at pH 3.0 and acetate at pH 4.6 were maintained formulations pH at T=0. However, an increase in pH of all prepared formulations were observed after storing at 60° C. or 2 weeks.

FIG. 28 is a graph showing fluctuations of pH of Naloxone formulations stored at 60° C. for two weeks comparing citrate buffer to acetate buffer. No changes or fluctuations in pH and osmolality were observed for naloxone placebo formulations stored at 60° C. for 2 weeks.

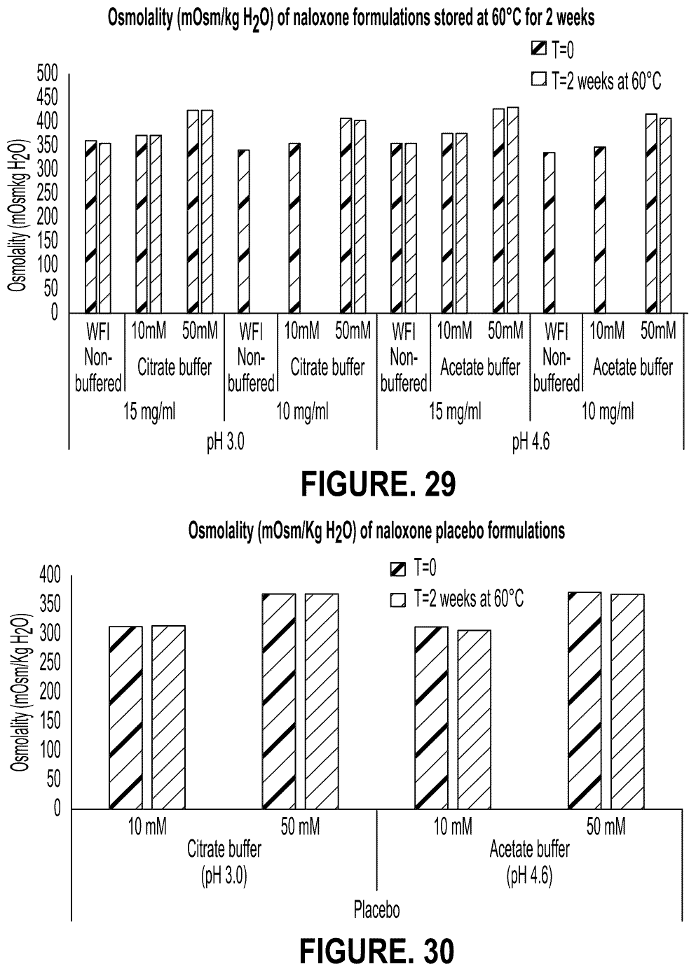

FIG. 29 is a graph showing the osmolality of Naloxone formulations in various buffers after storing at 60° C. for 2 weeks. Osmolality of naloxone intramuscular injection formulations was measured using osmometer (Advanced® Instruments, Model 3D3). All naloxone formulations prepared in non-buffered, citrate and acetate buffers at pH 3.0 and 4.6 were found to be hypertonic in nature and it ranges from 310 to 428 mOsm/Kg H2O. Higher values of osmolality were observed with increase in buffer strength at T=0. This wider range of osmolality is acceptable and tolerated due to rapid dilution with blood after injection. No changes in osmolality were observed for naloxone formulations stored at 60° C. or 2 weeks.

FIG. 30 is a graph showing the osmolality of Naloxone formulations in citrate and acetate buffers at pH 3.0 and 4.6 respectively, after storing at 60° C. for 2 weeks. It is noted that there is a slight decrease in osmolality for both concentrations of acetate buffer formulation at two weeks compared to the citrate buffer formulations.

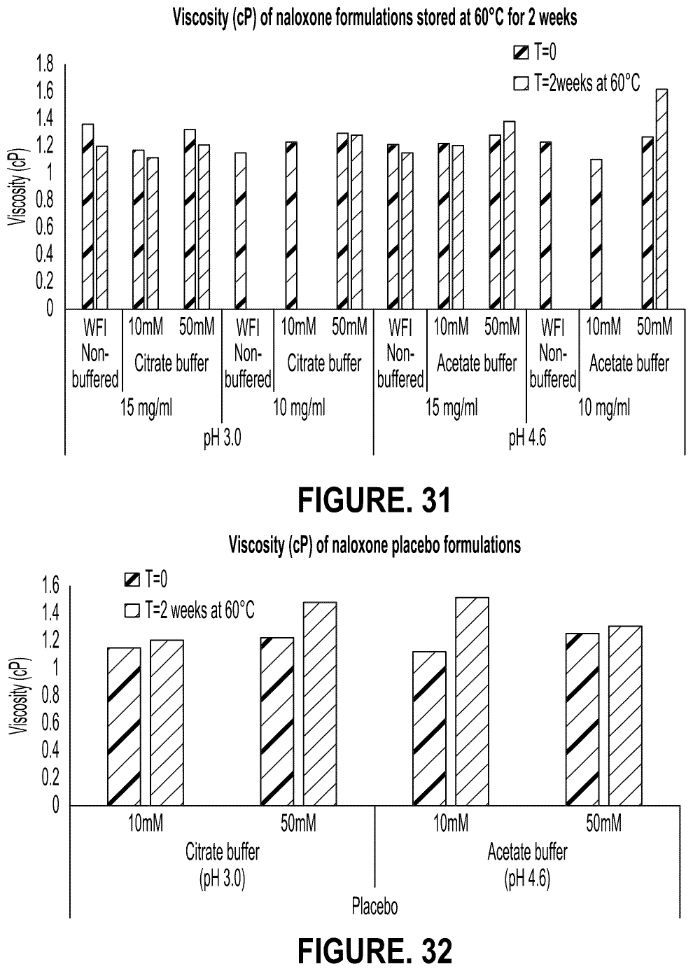

FIG. 31 is a graph showing the viscosity of Naloxone formulations after storing at 60° C. for 2 weeks. Viscosity of naloxone formulations prepared at 15 mg/ml and 10 mg/ml concentration was measured using a Brookfield Viscometer DV3T-LV (Brookfield Ametek, MA). The test was performed by immersing a spindle (CP-40) in 0.5 ml volume of formulation and rotated at a constant shear rate of 375 sec-1 and the viscosity was measured from the torque required to rotate the spindle at 50 rpm. The viscosity of formulations was measured at 25° C. temperature and the viscosity of formulations was ranging from 1.10 to 1.36 cP. The viscosity results revealed that these formulations can be easily injected. No significant changes in viscosity were observed in formulations stored 60° C. for 2 weeks.

FIG. 32 is a graph showing the viscosity of Naloxone formulations with a citrate buffer compared to an Acetate buffer after storing at 60° C. for 2 weeks. Slight increase in viscosity was observed for naloxone placebo formulations after storing at 60° C. for 2 weeks.

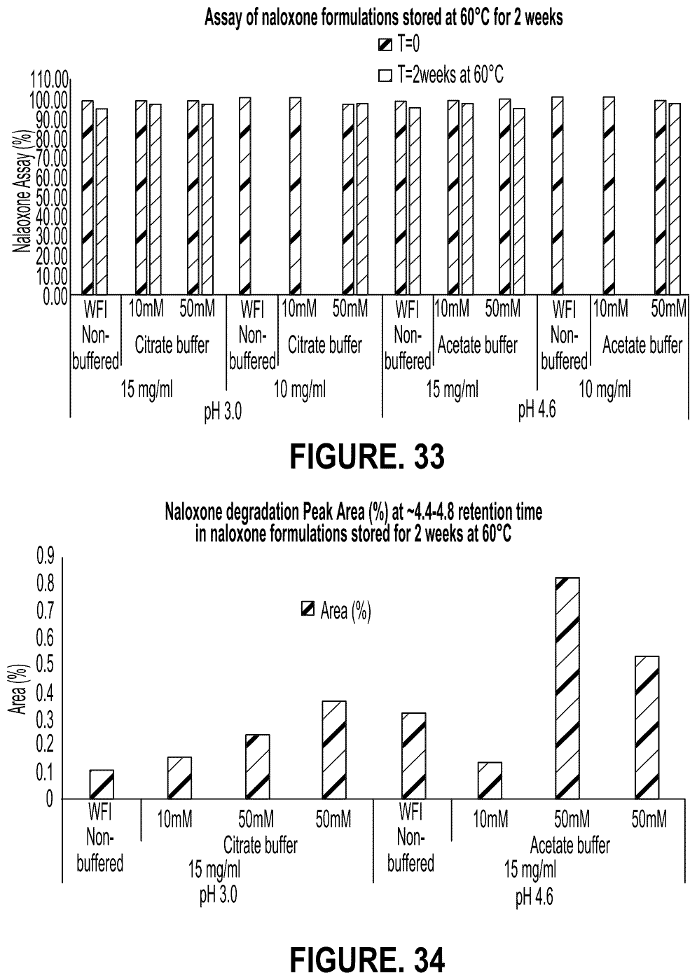

FIG. 33 is a graph showing an assay of Naloxone formulations after storing at 60° C. for 2 weeks in citrate buffer and acetate buffer at varying doses of Naloxone. Naloxone concentration in prepared formulations was determined using an HPLC analysis method. All formulations showed naloxone assay results within 95-105%. The assay of stored naloxone formulations at 60° C. for 2 weeks was also found to be within 95-105%.

FIG. 34 is a graph showing the presence of Naloxone degradation peaks in formulation Naloxone buffed with citrate compared to acetate buffer at various concentrations of each buffer. The Naloxone formulations were stored for 2 weeks at 60° C. Notably, Naloxone degradation was higher in formulations at 50 mM than those at 10 mM.

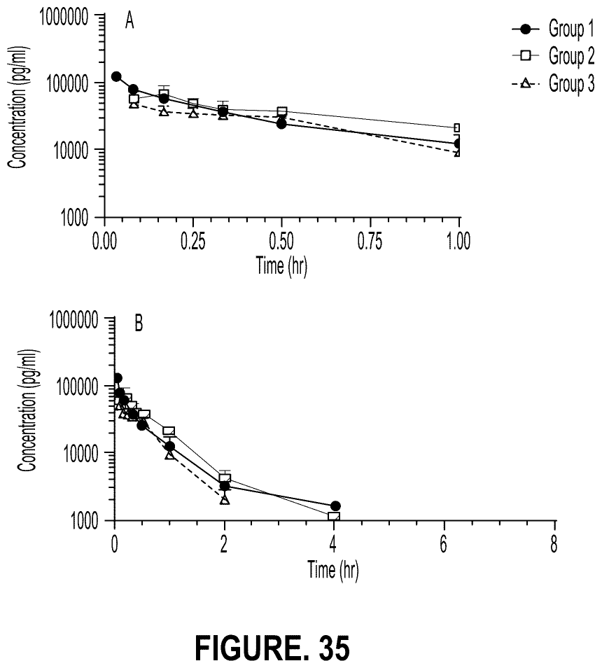

FIG. 35 is a graph showing the plasma concentrations of Naloxone after a single dose of 0.27 mg/kg administered amongst three groups of male beagle dogs. Administration was separated into three groups. Administered IV in Formulation 1 (Group 1), IM in Formulation 1 (Group 2) or IM in Formulation 2 (Group 3) to Male Beagle Dogs. A. Graph of Early Timepoints from 0.033 to 1 hr. B. Graph of Entire Time Course from 0.033 to 8 hr. Blood samples were collected from female dogs, n=3, and processed to plasma at 0, 0.033, 0.083, 0.167, 0.333, 0.5, 1, 2, 4, and 8 hr in the IV group and at 0, 0.083, 0.167, 0.25, 0.5, 1, 2, 4, and 8 hr in the IM dose groups. Plasma samples containing naloxone concentrations greater than LLOQ are plotted.

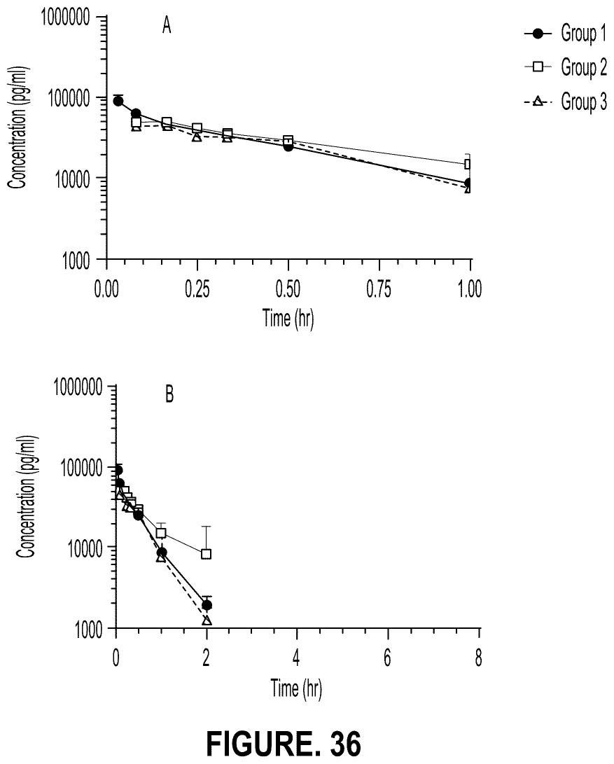

FIG. 36 is a graph showing the plasma concentrations of Naloxone after a single dose of 0.27 mg/kg administered amongst three groups of female beagle dogs. Administered IV in Formulation 1 (Group 1), IM in Formulation 1 (Group 2) or IM in Formulation 2 (Group 3) to Female Beagle Dogs. A. Graph of Early Timepoints from 0.033 to 1 hr. B. Graph of Entire Time Course from 0.033 to 8 hr. Blood samples were collected from female dogs, n=3, and processed to plasma at 0, 0.033, 0.083, 0.167, 0.333, 0.5, 1, 2, 4, and 8 hr in the IV group and at 0, 0.083, 0.167, 0.25, 0.5, 1, 2, 4, and 8 hr in the IM dose groups. Plasma samples containing naloxone concentrations greater than LLOQ are plotted.

### EXAMPLES

### Example 1

Initial pH Studies For pH stability studies, Naloxone hydrochloride was dissolved in a wide range of prepared buffers (Table 1) and analyzed after storing at desired pH for one week. Buffers are used in formulation to maintain pH of the drug substance and are very common in parenteral formulations of drugs that exhibit high aqueous solubility. Buffers used in intramuscular formulations range from 1-100 mM, but lower molar strengths are known to cause less myotoxicity resulting in less “pain” at the site of injection (Napaporn et al., Pharm. Dev. Technol. 5(1): 123-30 (2000). For the purpose of these initial pH stability experiment, a buffer strength of 10 mM was chosen. Buffers tested ranged from pH 3.0-10.6. Intramuscular formulations below pH 3.0 are extremely rare, owing to excessive acidity which can cause a burning sensation. Table 1 lists the various buffer systems used for this study.

TABLE 1
Buffer Systems used for pH Stability of Naloxone Hydrochloride

| S. No. | Buffer (10 mM) | pH |
|---|---|---|
| 1 | Citric acid/sodium citrate | 3.0 |
| 2 | Acetic acid/sodium citrate | 4.6 |
| 3 | Citric acid/sodium citrate | 5.0 |
| 4 | Potassium phosphate monobasic/dibasic | 6.5 |
| 5 | Sodium phosphate monobasic/dibasic | 7.4 |
| 6 | Glycine-sodium hydroxide | 10.0 |
| 7 | Carbonate-bicarbonate | 10.6 |
| 8 | Water | ~5.0 |

Buffers were created and mixed to a concentration of 10 mM with the pH stabilized. In example 1, Naloxone hydrochloride was dissolved in a wide range of buffers and analyzed after storing at a desired pH for one week. Buffers tested ranged from pH 3.0-10.6. Intramuscular formulations below pH 3.0 are rare due to discomfort associated with the injection site.

### Example 2

TABLE 2
List of Excipients and their Function

| Excipient Function | Excipient | Manufacturer |
|---|---|---|
| Buffering agents | Citric acid monohydrate | Spectrum Chemicals |
| | Trisodium citrate | |
| | dihydrate | |
| | Sodium acetate | |
| | trihydrate | |
| | Acetic acid, glacial | Mallinckrodt |
| Tonicity agents/modifier | Sodium chloride | Spectrum Chemicals |
| Preservatives | Methylparaben | Spectrum Chemicals |
| | Propylparaben | |
| | Benzalkonium chloride | Sigma Aldrich |
| Vehicle | Water for injection | Baxter |

Samples prepared for pH-Stability of naloxone with and without buffering agents (citrate and acetate at pH 3.0 and pH 4.6 respectively) under nitrogen gas overlay were left at 40° C.±2° C./75%±5% relative humidity (RH) and at 60° C. for a total of 5 weeks for this excipient compatibility study. A stability storage chamber was used for 40° C.±2° C./75%±5% while a regular oven was used for the 60° C. samples. Briefly, naloxone samples were prepared in 20 ml of water for injection or freshly prepared 10 mM citrate buffer and/or acetate buffer in 25 ml volumetric flask. To this, approximately 375 mg naloxone hydrochloride dihydrate was added and vortex mixed until drug dissolves. The pH of the naloxone solutions was adjusted to 3.0 and 4.6 with 0.1N HCl solution. Samples were diluted up to the 25 ml mark with water for injection or citrate buffer pH 3.0 or acetate buffer pH 4.6. These naloxone solutions were filtered using 0.22 μm, 13 mm, PVDF (Pall scientific) syringe filters and pH of the filtrate was measured. The filtered solution was aliquoted into 2.0 ml clear, sterile, Type-I glass vials and closed with 13 mm red rubber closures crimped with aluminum blue flip cap seals. All vials were overlaid with nitrogen gas (filtered with 0.22u PTFE, Acro®50 Vent In-Line filter, Pall scientific) before sealing and stored at 40° C.±2° C./75%±5% relative humidity (RH) or 60° C. for 5 weeks. These samples were evaluated for pH, assay and impurities of naloxone at intervals of T=0, 1 week and T=5 weeks. To evaluate interaction of tonicity agent (sodium chloride at 5% w/V, hypertonic) and preservatives (0.2% methylparaben, 0.025% propylparaben, and 0.02% benzalkonium chloride) with naloxone, another set of samples were prepared as described above and stored at 60° C. for T=5 weeks. The concentration of sodium chloride for excipient compatibility study was selected based on its solubility in naloxone hydrochloride solution. Similarly, the concentrations of preservatives were selected based on their solubility in water as well as their maximum potency (%) in per unit dose of intramuscular injection in FDA's previously approved products. The selected buffering agents, tonicity agent and preservatives were all listed in FDA's Inactive Ingredient Guide (IIG). These samples were analyzed at T=2 weeks and T=5 weeks for pH, assay and impurities for naloxone. Physical appearance of samples with sodium chloride and with preservatives was visually observed and photographed on T=0 day and T=1, 2, and 4 weeks.

### Example 3

Samples prepared with additional excipients

TABLE 3
Samples Placed on Excipient Compatibility Study

| Buffer | Formulation # | Excipients |
|---|---|---|
| Non-buffered solution | 16803-20-1 | WFI. pH 3.0 |
| (pH adjusted with | 16803-20-2 | WFI. pH 4.6 |
| 0.1 NHCl) | 16803-30-1 | WFI. 5% NaCl, pH 3.0 |
| | 16803-30-2 | WFI, 5% NaCl, H 4.6 |
| | 16803-31-1 | WFI, Methylparaben, propylparaben pH 3.0 |
| | 16803-31-2 | WFI. Methylparaben, propylparaben pH 4.6 |
| | 16803-31-5 | WFI, Benzalkonium chloride. pH 3.0 |
| | 16803-31-6 | WFI, Benzalkonium chloride, pH 4.6 |
| Buffered solution | 16803-20-3 | Citric acid. Trisodium citrate dihydrate, Milli-Q water, |
| (10 mM) | | pH 3.0 |
| | 16803-20-4 | Acetic acid, Sodium acetate trihydrate, Milli-Q water. |
| | | pH 4.6 |
| | 16803-30-3 | Citric acid, Trisodium citrate dihydrate, 5% NaCL Milli- |
| | | Q water, pH 3.0 |
| | 16803-30-4 | Acetic acid. Sodium acetate trihydrate, 5% NaCl, |
| | | Milli-Q water, pH 4.6 |
| | 16803-31-3 | Methylparaben. propylparaben. Citric acid. Trisodium |
| | | citrate dihydrate, Milli-Q water, pH 3.0 |
| | 16803-31-4 | Methylparaben, propylparaben. Acetic acid, Sodium |
| | | acetate trihydrate, Milli-Q water, pH 4.6 |
| | 16803-31-7 | Citric acid, Trisodium citrate dihydrate, Benzalkonium |
| | | chloride. Milli-Q water, pH 3.0 |
| | 16803-31-8 | Acetic acid, Sodium acetate trihydrate, Benzalkonitu11 |
| | | chloride. Milli-Q water. pH 4.6 |

### Results

The pH and assay of naloxone for excipient compatibility samples stored at 40° C.±2° C./75%±5% RH and 60° C. on T=0-day, 1 week and T=5 weeks were evaluated.

Physical appearance: Based on visual observation, all naloxone samples prepared in 10 mM buffered and non-buffered solutions with buffering agents, at 40° C.±2° C./75%±5% RH and in 60° C. for 5 weeks were clear and with tonicity agent and with preservatives were also clear and no discoloration was observed after storing at 60° C. for 2 weeks.

pH: The pH fluctuations of naloxone HCl solution prepared with and without buffering agents and stored at 40° C.±2° C./75%±5% RH chamber and at 60° C. Similarly, the pH fluctuations of naloxone prepared in buffering and a non-buffering solution with tonicity agents and with preservatives stored at 60° C. An increase in pH was observed for naloxone prepared at 15 mg/ml strength in non-buffered solution at pH 4.6 (pH was adjusted with 0.1N HCl) after storing at 40° C.±2° C./75% 5% RH and at 60° C. for 5 weeks, and the naloxone samples containing buffering agents (citric acid, sodium citrate, sodium acetate, acetic acid) did not show much variation in pH after storing at 40° C.±2° C./75%±5% RH and at 60° C. for T=1 week and T=5 weeks.

### Example 4

### Other Commercial Intramuscular Naloxone Formulation Details:

TABLE 4
Other Commercial Intramuscular Naloxone Formulation

| Product | | | | |
|---|---|---|---|---|
| Name | Manufacturer | Route | Strength | pH |
| Evizio ® | Kaleo | IM, SC | 0.4 mg (1.0 | 3.0-4.5 |
| | | (Autoinjector) | mg/ml)* | |
| | Kaleo | | 2.0 mg (5.0 | 3.0-4.5 |
| | | | mg/ml)* | |
| | Hameln | IM, IV | 0.4 mg/ml | 3.1-4.5 |
| Generic | Mylan | IM, SC, IV | 0.4 mg/ml | 3.0-6.5 |

*Evzio Autoinjector single dose is 0.4 ml

### Example 5

### Characterization Studies

TABLE 5
Characterization of Results of Naloxone Formulations

| | | | | | | | Spcific | HPLC | |
|---|---|---|---|---|---|---|---|---|---|
| Formulation | | Naloxone | | Buffer | | Osmolality | Gravity | Assay | Viscosity |
| # | pH | Concentration | Buffer | Strength | pH | (mOsm · kgH₂O) | (g/cm³) | (%) | (cP) |
| 16803-40-1 | 3.0 | 15 mg/ml | NA | WFI | 3.03 | 363 | 1.004 | 99.33 | 1.36 |
| 16803-43-1 | | | Citrate | 10 mM | 3.03 | 372 | 1.002 | 100.00 | 1.17 |
| 16803-47-1 | | | buffer | 50 mM | 2.96 | 425 | 1.003 | 99.73 | 1.32 |
| 16803-40-2 | | 10 mg/ml | NA | WFI | 3.06 | 341 | 1.003 | 101.00 | 1.15 |
| 16803-43-2 | | | Citrate | 10 mM | 3.16 | 354 | 1.003 | 100.00 | 1.23 |
| 16803-49-1 | | | buffer | 50 mM | 2.82 | 406 | 1.003 | 98.20 | 1.29 |
| 16803-40-3 | 4.6 | 15 mg/ml | NA | WFI | 4.6 | 357 | 1.003 | 98.67 | 1.21 |
| 16803-44-1 | | | Acetate | 10 mM | 4.57 | 377 | 1.003 | 100.00 | 1.22 |
| 16803-47-2 | | | buffer | 50 mM | 4.6 | 428 | 1.004 | 100.40 | 1.28 |
| 16803-40-4 | | 10 mg/ml | NA | WFI | 4.57 | 337 | 1.002 | 101.00 | 1.23 |
| 16803-44-2 | | | Acetate | 10 mM | 4.61 | 346 | 1.002 | 100.00 | 1.10 |
| 16803-49-2 | | | buffer | 50 mM | 4.54 | 416 | 1.003 | 99.50 | 1.26 |
| 16803-51-1 | 3.0 | Placebo | Citrate | 10 mM | 3.14 | 313 | 1.003 | — | 1.16 |
| 16803-51-2 | | | buffer | 50 mM | 3.06 | 368 | 1.004 | — | 1.23 |
| | | | (pH 3.0) | | | | | | |
| 16803-51-3 | 4.6 | | Acetate | 10 mM | 4.57 | 310 | 1.003 | — | 1.13 |
| 16803-51-4 | | | Buffer | 50 mM | 4.67 | 371 | 1.003 | — | 1.25 |
| | | | (pH 4.6) | | | | | | |

NA = Non-buffered
WFI = Water For Injection

### Characterization Studies

Characterization results for naloxone and corresponding placebo formulations at T=0 are presented in Example 5.

Physical appearance: All naloxone formulations at 15 mg/ml and 10 mg/ml prepared in 10 mM and 50 mM citrate and acetate buffers at pH 3.0 and 4.6 were clear at T=0. Naloxone formulations at 15 mg/ml and 10 mg/ml strength prepared in nonbuffered solutions at pH 4.6 and in acetate buffer at 10 mM and 50 mM buffer strength changed color from clear to light yellow after storing at 60° C. for T=1 week and this discoloration was increased with time T=2 weeks. However, samples prepared at 15 mg/ml and 10 mg/ml strength prepared in nonbuffered solutions at pH 3.0 and in citrate buffer at 10 mM and 50 mM buffer strength were all clear and no discoloration was observed after storing at 60° C. for 2 weeks. This discoloration of naloxone formulation is attributed to be related to naloxone drug substance as placebo formulations were clear after storing at 60° C. for 2 weeks.

pH: The pH of the prepared naloxone formulations was measured using PH meter (Model 340, Corning). Based on the pH results of formulations, buffer strengths 10 mM and 50 mM for citrate at pH 3.0 and acetate at pH 4.6 were maintained formulations pH at T=0. However, an increase in pH of all prepared formulations were observed after storing at 60° C. for 2 weeks. A comparative plot of pH fluctuations in naloxone formulations is presented in FIG. 27.

Osmolality: Osmolality of naloxone intramuscular injection formulations was measured using osmometer (Advanced® Instruments, Model 3D3). All naloxone formulations prepared in nonbuffered, citrate and acetate buffers at pH 3.0 and 4.6 were found to be hypertonic in nature and it ranges from 310 to 428 mOsm/Kg H2O. Higher values of osmolality were observed with increase in buffer strength at T=0. This wider range of osmolality is acceptable and tolerated due to rapid dilution with blood after injection. No changes in osmolality were observed for naloxone formulations stored at 60° C. for 2 weeks. A comparative plot of osmolality of naloxone formulations at T=0 and T=2 weeks at 60° C. is presented in FIG. 29.

Density (specific gravity): Density or specific gravity of naloxone formulations were measured using pycnometer (Specific gravity, Method 1, USP<841>). All naloxone formulations prepared at 15 mg/ml and 10 mg/ml showed density of around 1.002 to 1.004 g/cm³. Density of naloxone formulation stored in crimped vials at 60° C. was not determined due to lack of sufficient volume of formulation for testing using 10 ml volume pycnometer.

Viscosity: Viscosity of naloxone formulations prepared at 15 mg/ml and 10 mg/ml concentration was measured using a Brookfield Viscometer DV3T-LV (Brookfield Ametek, MA). The test was performed by immersing a spindle (CP-40) in 0.5 ml volume of formulation and rotated at a constant shear rate of 375 sec-1 and the viscosity was measured from the torque required to rotate the spindle at 50 rpm. The viscosity of formulations was measured at 25° C. temperature and the viscosity of formulations was ranging from 1.10 to 1.36 cP. The viscosity results revealed that these formulations can be easily injected. No significant changes in viscosity were observed in formulations stored 60° C. for 2 weeks. A comparative plot of formulations viscosities at T=0 and T=2 weeks 60° C. is presented in FIG. 31.

Naloxone Assay: Naloxone concentration in prepared formulations was determined using a HPLC method. All formulations showed naloxone assay results within 95-105%. The assay of stored naloxone formulations at 60° C. for 2 weeks was also found to be within 95-105%. A comparative plot of naloxone formulations at T=0 and T=2 weeks storage at 60° C. is presented in FIG. 33. The chromatograms at T=0 of prepared naloxone formulations at 15 mg/ml and 10 mg/ml concentration at 10 mM and 50 mM buffer strength in citrate, acetate and nonbuffered did not show any additional peaks (>0.1% peak area) at ˜4.4 to 4.8 minutes. However, the chromatograms of naloxone formulations stored at 60° C. for 2 weeks showed an additional degradation peak at ˜4.4 to 4.8 minutes (circled in chromatograms below FIGS. 11 to 15, and 25). A comparative plot for percent area peak for these samples is presented in FIG. 34.

Conclusions: Naloxone formulations prepared at 15 mg/ml and 10 mg/ml concentration in 10 mM and 50 mM citrate buffer at pH 3.0 were found to be visually clear after storing at extreme conditions of 60° C. for 2 weeks. No difference in pH, density, and viscosity was observed between 10 mg/ml and 15 mg/ml naloxone concentration formulations prepared in nonbuffered, buffered solutions. Higher osmolality values were observed in naloxone formulations prepared in both citrate and acetate buffers prepared at 50 mM buffer strength. Stressing of naloxone formulations at extreme conditions (60° C.) for 2 weeks revealed that naloxone formulations prepared in nonbuffered and acetate buffer solutions at pH 4.6 were not physically stable. For pilot batch preparation, two formulations at 10 mg/ml and 15 mg/ml naloxone concentration in 10 mM citrate buffer at pH 3.0 were selected. Otherwise, one formulation at one single concentration at 10 mM buffer strength and one backup at 50 mM buffer strength were selected.

### Example 6

### Photostability Studies

All samples were placed in a photostability chamber flat in order to provide maximum exposure to a cool white fluorescent and near UV lamp until>1.2 million lux hours and >200 watt hours square meter.

TABLE 6
Naloxone Related Substances/Impurity Levels
After Exposure to 1.2 × 10⁶ Lux Hours

| | Ph. Eur. | USP | | |
|---|---|---|---|---|
| | Acceptance | Acceptance | Citrate Buffer Formulation | |
| Test Performed | Criteria | Criteria | Without Carton | With Carton |
| Appearance | | | | |
| pH | | | 3.28 ± 0.01 | 3.25 ± 0.02 |
| Osmolarity | | | 0.358 ± 0.1 | 0.357 ± 0.01 |
| (mOsm/ml) | | | | |
| Assay | | | 98.70% | 100.20% |
| Related Substances | | | | |
| Noroxymorphone | NMT 0.2% | Not included | 0.05% | No peak |
| (Ph. Eur. Impurity A) | | | | |
| 3-0-Allylnaloxone | NMT 0.2% | | No peak | No peak |
| (Ph. Eur. Impurity B) | | | | |
| 10-α- | NMT 0.2% | | 0.29% | 0.06% |
| Hydroxynaloxone | | | | |
| (Ph. Eur. Impurity C) | | | | |
| 7,8- | NMT 0.30% | | No peak | No peak |
| Didehydronaloxone | | | | |
| (Ph. Eur, Impurity D) | | | | |
| 2,2′-Bisnaloxone | NMT 0.2% | NMT 4.0% | 0.43% | No peak |
| (Ph. Eur. Impurity E) | | | | |
| 10-β- | NMT 0.2% | Not included | No peak | No peak |
| Hydroxynaloxone | | | | |
| (Ph. Eur. Impurity F) | | | | |
| Specified Peak (RRT | NMT 0.35% | | 0.03% | No peak |
| 0.51) | | | | |
| Specified Peak (RRT | NMT 0.35% | | 0.23% | No peak |
| 0.61) | | | | |
| Unspecified Peaks | NMT 0.1% | | No peak | No peak |

Formulation 16847-12-1: Naloxone HCL dihydrate 10 mg/mL; citric acid monohydrate; trisodium citrate dihydrate; sodium chloride; methylparaben; propylparaben; water; hydrochloric acid/sodium hydroxide for pH adjustment; pH 3.0

### Example 7

### 6 Month Thermostability Studies of High-Dose Naloxone Formulations

TABLE 7
6 Month Thermostability studies of High-dose Naloxone Formulations

| | | | Storage Conditions | | | |
|---|---|---|---|---|---|---|
| | | | | | | 25° C. ± 2° C./ |
| | Acceptance | | 5° C. ± 3° C. | | | 60% ± 5% RH |
| Test Performed | Criteria | Initial | 1 M | 3 M | 6 M | 1 M |
| Appearance | Clear | Clear | Clear | Clear | Clear | Clear |
| pH | 3.0 to 3.5 | 3.10 | 3.18 | 3.11 | 3.08 | 3.18 |
| Osmolarity | 0.330 to | 0.358 | 0.356 | 0.356 | 0.356 | 0.354 |
| (mOsm/mL) | 0.360 | | | | | |
| Assay | 90 to 110% | 100.43 | 98.73 | 99.10 | 99.70 | 98.80 |
| Related Substances | | | | | | |
| Noroxymorphone | NMT 0.2% | No | 0.01% | 0.01% | No | 0.02% |
| (Ph. Eur. Impurity A) | | peak | | | peak | |
| 3-0-Allylnaloxone | NMT 0.2% | No | No | No | No | No |
| (Ph. Eur. Impurity B) | | peak | peak | peak | peak | peak |
| 10-α- | NMT 0.2% | 0.05% | 0.06% | 0.07% | 0.05% | 0.07% |
| Hydroxynaloxone | | | | | | |
| (Ph. Eur. Impurity C) | | | | | | |
| 7,8- | NMT 0.3% | No | No | No | No | No |
| Didehydronaloxone | | peak | peak | peak | peak | peak |
| (Ph. Eur. Impurity D) | | | | | | |
| 2,2′-Bisnaloxone | NMT 0.2% | No | 0.02% | 0.03% | No | 0.02% |
| (Ph. Eur. Impurity E) | | peak | | | peak | |
| 10-β- | NMT 0.2% | No | No | No | No | No |
| Hydroxynaloxone | | peak | peak | peak | peak | peak |
| (Ph. Eur. Impurity F) | | | | | | |
| Specified Peak (RRT | NMT 0.35% | No | No | No | No | No |
| 0.51) | | peak | peak | peak | peak | peak |
| Specified Peak (RRT | NMT 0.35% | No | 0.01% | 0.03% | No | 0.03% |
| 0.61) | | peak | | | peak | |
| Specified Peak (RRT | NMT 0.1% | No | No | No | No | No |
| 0.32) | | peak | peak | peak | peak | peak |
| Specified Peak (RRT | NMT 0.1% | No | No | No | No | No |
| 0.58) | | peak | peak | peak | peak | peak |
| Specified Peak (RRT | NMT 0.1% | No | No | 0.01 | No | No |
| 0.71) | | peak | peak | | peak | peak |
| Total Relates | NMT 1.2% | 0.05% | 0.10% | 0.15% | 0.05% | 0.13% |
| Substances | | | | | | |
| Preservative Content | | | | | | |
| Methylparaben | 90% to 110% | 99.75 | 101.37 | 100.67 | 100.43 | 101.37 |
| Propylparaben | 90 to 110% | 100.37 | 99.67 | 100.23 | 100.3 | 99.07 |
| 4-hydroxybenzoic | NMT 75 | 4.73 | 4.43 | 4.93 | 5.25 | 5.83 |
| acid (μg/mL) | | | | | | |

| | Storage Conditions | | | | |
|---|---|---|---|---|---|
| | 25° C. ± 2° C./ | | 40° C. ± 2° C./ | | |
| | 60% ± 5% RH | | 75% ± 5% RH | | |
| Test Performed | 3 M | 6 M | 1 M | 3 M | 6 M |
| Appearance | Clear | Clear | Clear | Clear | Clear |
| pH | 3.11 | 3.04 | 3.18 | 3.05 | 3.04 |
| Osmolarity | 0.356 | 0.357 | 0.354 | 0.354 | 0.356 |
| (mOsm/mL) | | | | | |
| Assay | 99.03 | 99.53 | 98.60 | 98.97 | 98.57 |
| Related Substances | | | | | |
| Noroxymorphone | 0.02% | No | 0.03% | 0.07% | 0.12% |
| (Ph. Eur. Impurity A) | | peak | | | |
| 3-0-Allylnaloxone | No | No | No | No | No |
| (Ph. Eur. Impurity B) | peak | peak | peak | peak | peak |
| 10-α- | 0.08% | 0.07% | 0.08% | 0.10% | 0.11% |
| Hydroxynaloxone | | | | | |
| (Ph. Eur. Impurity C) | | | | | |
| 7,8- | No | No | No | No | No |
| Didehydronaloxone | peak | peak | peak | peak | peak |
| (Ph. Eur. Impurity D) | | | | | |
| 2,2′-Bisnaloxone | 0.04% | 0.05% | 0.07% | 0.16% | 0.18% |
| (Ph. Eur. Impurity E) | | | | | |
| 10-β- | No | No | No | No | No |
| Hydroxynaloxone | peak | peak | peak | peak | peak |
| (Ph. Eur. Impurity F) | | | | | |
| Specified Peak (RRT | 0.02% | No | 0.04% | 0.15% | 0.30% |
| 0.51) | | peak | | | |
| Specified Peak (RRT | 0.07% | 0.09% | 0.11% | 0.24% | 0.28% |
| 0.61) | | | | | |
| Specified Peak (RRT | 0.01% | No | No | 0.03% | 0.05% |
| 0.32) | | peak | peak | | |
| Specified Peak (RRT | No | No | No | No | No |
| 0.58) | peak | peak | peak | peak | peak |
| Specified Peak (RRT | 0.02% | No | No | 0.05% | 0.07% |
| 0.71) | | peak | peak | | |
| Total Relates | 0.25% | 0.21% | 0.33% | 0.81% | 1.11% |
| Substances | | | | | |
| Preservative Content | | | | | |
| Methylparaben | 100.87 | 100.03 | 101.07 | 100.37 | 97.87 |
| Propylparaben | 100.50 | 99.13 | 98.97 | 99.77 | 99.90 |
| 4-hydroxybenzoic | 8.40 | 14.04 | 11.07 | 25.47 | 53.91 |
| acid (μg/mL) | | | | | |

NMT = Note more than
M = Month
RH = Relative Humidity
Clear/Colorless
Formulation 16847-12-1: Naloxone HCL dihydrate 10 mg/mL; citric acid monohydrate; trisodium citrate dihydrate; sodium chloride; methylparaben; propylparaben; water; hydrochloric acid/sodium hydroxide for pH adjustment; pH 3.0

### Example 8

### 24 Month Thermostability Studies of High-Dose Naloxone Formulations

TABLE 8
Naloxone Related substances/Impurity levels after
storing naloxone formulations for 24 months

| | | | | Storage condition-24 Months | |
|---|---|---|---|---|---|
| | Impurity/Related | EP | USP | | 25° C. ± 2° C./ |
| EP Impurity | substance name | Limits¹ | limits² | 5° C. ± 2° C. | 60% RH ± 5% RH |
| Impurity A | Noroxymorphone | NMT | Not | No peak | 0.05% |
| | | 0.2% | included | | |
| Impurity B | 3-O-Allyinaloxone | NMT | | No peak | No peak |
| | | 0.2% | | | |
| Impurity C | 10-α- | NMT | | 0.02% | 0.05% |
| | Hydroxynaloxone | 0.2% | | | |
| Impurity D | 7,8- | 0.30% | | No peak | No peak |
| | Didehydronaloxone | | | | |
| Impurity E | 2,2′-Bisnaloxone | NMT | NMT | 0.01% | 0.09% |
| | | 0.2% | 4.0% | | |
| Impurity F | 10-β- | NMT | Not | No peak | No peak |
| | Hydroxynaloxone | 0.2% | included | | |
| | Specified-1 (RRT | NMT | | 0.01% | 0.07% |
| | 0.51) | 0.35% | | | |
| | Specified-2 (RRT | NMT | | 0.01% | 0.14% |
| | 0.61) | 0.35% | | | |
| | Unspecified-3 (RRT | NMT | | No peak | No peak |
| | 0.32) | 0.1% | | | |
| | Unspecified-4 (RRT | NMT | | No peak | No peak |
| | 0.58) | 0.1% | | | |
| | Unspecified-5 (RRT | NMT | | No peak | No peak |
| | 0.71) | 0.1% | | | |

¹Naloxone hydrochloride dihydrate drug substance monograph; EUROPEAN PHARMACOPOEIA 6.0
²Naloxone hydrochloride injection; USP monograph
Formulation 16847-12-1: Naloxone HCL dihydrate 10 mg/mL; citric acid monohydrate; trisodium citrate dihydrate; sodium chloride; methylparaben; propylparaben; water; hydrochloric acid/sodium hydroxide for pH adjustment; pH 3.0

### Example 9

### 24 Month Thermostability Studies of High-Dose Naloxone Formulations

TABLE 9
24 Month Thermostability studies of High-dose Naloxone Formulations

| Storage | | | Osmolality | Density | Viscosity | Assay | |
|---|---|---|---|---|---|---|---|
| Condition | Duration | pH | (mOsmol/kg) | (g/ml) | (cP) | (mg/ml) | HPLC assay (%) |
| RT | T = 0 | 3.10 ± 0.00 | 357.67 ± 0.58 | 1.01 ± 0.00 | 1.19 ± 0.03 | 10.04 ± 0.04 | 100.43 ± 0.35 |
| 5° C. ± 2° C. | 1-Month | 3.18 ± 0.00 | 356.00 ± 1.00 | 0.99 ± 0.00 | 1.22 ± 0.04 | 9.87 ± 0.02 | 98.73 ± 0.15 |
| | 3-Months | 3.11 ± 0.01 | 355.67 ± 0.58 | 1.01 ± 0.01 | 1.15 ± 0.03 | 9.91 ± 0.01 | 99.10 ± 0.10 |
| | 6-Months | 3.08 ± 0.01 | 355.67 ± 0.58 | 1.01 ± 0.00 | 1.14 ± 0.02 | 9.97 ± 0.02 | 99.70 ± 0.20 |
| | 9-Months | 2.99 ± 0.02 | 358.00 ± 0.58 | 1.01 ± 0.01 | 1.33 ± 0.01 | 10.02 ± 0.01 | 100.23 ± 0.06 |
| | 12-Months | 3.05 ± 0.01 | 359.00 ± 1.00 | 1.01 ± 0.00 | 1.28 ± 0.03 | 9.96 ± 0.01 | 99.63 ± 0.06 |
| | 18-Months | 2.86 ± 0.07 | 356.00 ± 1.15 | 1.01 ± 0.00 | 1.35 ± 0.05 | 10.01 ± 0.01 | 100.10 ± 0.10 |
| | 24-Months | 2.98 ± 0.04 | 358.67 ± 0.58 | 1.02 ± 0.01 | 1.33 ± 0.04 | 9.99 ± 0.00 | 99.90 ± 0.10 |
| 25° C. ± 2° C./ | 1-Month | 3.18 ± 0.01 | 354.00 ± 1.00 | 0.99 ± 0.00 | 1.18 ± 0.05 | 9.88 ± 0.02 | 98.80 ± 0.17 |
| 60% RH ± 5% | 3-Months | 3.11 ± 0.01 | 355.67 ± 0.58 | 1.01 ± 0.00 | 1.09 ± 0.06 | 9.90 ± 0.01 | 99.03 ± 0.12 |
| RH | 6-Months | 3.04 ± 0.01 | 357.33 ± 0.58 | 1.00 ± 0.00 | 1.17 ± 0.04 | 9.95 ± 0.02 | 99.53 ± 0.15 |
| | 9-Months | 2.92 ± 0.04 | 358.00 ± 0.58 | 1.01 ± 0.01 | 1.26 ± 0.05 | 9.97 ± 0.01 | 99.70 ± 0.10 |
| | 12-Months | 3.04 ± 0.01 | 359.00 ± 0.58 | 1.02 ± 0.00 | 1.24 ± 0.01 | 9.96 ± 0.01 | 99.57 ± 0.06 |
| | 18-Months | 2.81 ± 0.02 | 358.00 ± 0.00 | 1.01 ± 0.01 | 1.30 ± 0.04 | 10.02 ± 0.02 | 100.20 ± 0.20 |
| | 24-Months | 2.98 ± 0.03 | 359.00 ± 1.00 | 1.01 ± 0.01 | 1.33 ± 0.04 | 10.01 ± 0.10 | 100.10 ± 0.70 |
| 40° C. ± 2° C./ | 1-Month | 3.18 ± 0.00 | 354.00 ± 0.00 | 1.00 ± 0.00 | 1.22 ± 0.02 | 9.86 ± 0.03 | 98.60 ± 0.26 |
| 75% RH ± 5% | 3-Months | 3.05 ± 0.01 | 354.00 ± 1.00 | 1.01 ± 0.00 | 1.22 ± 0.02 | 9.89 ± 0.05 | 98.97 ± 0.47 |
| RH | 6-Months | 3.04 ± 0.01 | 356.00 ± 1.00 | 1.01 ± 0.00 | 1.36 ± 0.06 | 9.86 ± 0.02 | 98.57 ± 0.15 |
| 60 C. Oven | 1-Month | 3.17 ± 0.01 | 356.00 ± 0.58 | 1.01 ± 0.00 | 1.20 ± 0.05 | 9.76 ± 0.05 | 97.63 ± 0.47 |
| | 3-Months | 3.01 ± 0.02 | 359.67 ± 1.15 | 1.00 ± 0.00 | 1.22 ± 0.07 | 9.67 ± 0.05 | 96.70 ± 0.52 |
| | 6-Months | 2.98 ± 0.01 | 359.67 ± 1.53 | 1.00 ± 0.00 | 1.16 ± 0.03 | 9.38 ± 0.02 | 93.77 ± 0.15 |

Specifications- pH: 3.0 to 3.5, Osmolality: 330 to 360 mOsmol/kg, Density: 1.00 ± 0.05 g/mL, Assay by HPLC: 90.0% to 110.0%
Formulation 16847-12-1: Naloxone HCL dihydrate 10 mg/mL; citric acid monohydrate; trisodium citrate dihydrate; sodium chloride; methylparaben; propylparaben; water; hydrochloric acid/sodium hydroxide for pH adjustment; pH 3.0

### Example 10

24 Month Preservative assays of High-dose Naloxone Formulations

TABLE 10
Preservative content assay by HPLC % in naloxone formulations after
storage for 24-months (Mean ± SD), (Specifications: 90-110%)

| | Condition | | Methylparaben (MP) | Propylparaben (PP) |
|---|---|---|---|---|
| 1-Month | Freeze-Thaw | | 101.17 ± 0.17 | 99.10 ± 0.36 |
| | 5° | C. | 101.37 ± 0.09 | 99.67 ± 0.17 |
| | 25° | C. | 101.37 ± 0.24 | 99.07 ± 0.59 |
| | 40° | C. | 101.07 ± 0.33 | 98.97 ± 0.52 |
| | 60° | C. | 98.47 ± 0.05 | 95.03 ± 0.17 |
| 3-Months | 5° | C. | 100.67 ± 0.09 | 100.23 ± 0.81 |
| | 25° | C. | 100.87 ± 0.09 | 100.50 ± 0.24 |
| | 40° | C. | 100.37 ± 0.45 | 99.77 ± 0.21 |
| | 60° | C. | 92.53 ± 0.33 | 88.93 ± 0.25 |
| 6-Months | 5° | C. | 100.43 ± 0.12 | 100.30 ± 0.22 |
| | 25° | C. | 100.03 ± 20.21 | 99.13 ± 0.57 |
| | 40° | C. | 97.87 ± 0.21 | 99.90 ± 0.67 |
| | 60° | C. | 82.13 ± 0.12 | 82.37 ± 0.42 |
| 9-Months | 5° | C. | 100.77 ± 0.05 | 101.77 ± 0.12 |
| | 25° | C. | 100.13 ± 0.09 | 100.70 ± 0.14 |
| 12-Months | 5° | C. | 100.37 ± 0.09 | 101.17 ± 0.12 |
| | 25° | C. | 100.57 ± 0.05 | 99.83 ± 0.05 |
| 18-Months | 5° | C. | 99.97 ± 0.17 | 100.73 ± 0.31 |
| | 25° | C. | 99,17 ± 0.19 | 100.00 ± 0.22 |
| 24-Months | 5° | C. | 100 ± 0.10 | 100.1 ± 0.00 |
| | 25° | C. | 98.4 ± 0.10 | 100.27 ± 0.06 |

Formulation 16847-12-1: Naloxone HCL dihydrate 10 mg/mL; citric acid monohydrate; trisodium citrate dihydrate; sodium chloride; methylparaben; propylparaben; water; hydrochloric acid/sodium hydroxide for pH adjustment; pH 3.0

### Example 11

Concentration of 4-Hydroxybenzoic Acid after Storing Naloxone Formulations for 24 Months.

TABLE 11
Concentration of 4-hydroxybenzoic acid (μg/ml) generated after storing of active
naloxone formulations for 24 Months (Mean ± SD; n = 3)

| | T = 0 | | | | | | |
|---|---|---|---|---|---|---|---|
| | 4.5 ± 0.00 | | | | | | |
| Condition | 1-Month | 3-Months | 6-Months | 9-Months | 12-Months | 18-Months | 24-Months |
| 5° C. ± 2° C. | 4.43 ± 0.05 | 4.93 ± 0.17 | 5.25 ± 0.02 | 5.22 ± 0.03 | 5.19 ± 0.02 | 5.63 ± 0.15 | 5.84 ± 0.02 |
| 25° C. ± | 5.83 ± 0.05 | 8.40 ± 0.00 | 14.04 ± 0.17 | 17.20 ± 0.06 | 19.58 ± 0.03 | 25.35 ± 0.04 | 34.26 ± 0.21 |
| 2° C./60% | | | | | | | |
| RH ± 5% | | | | | | | |
| RH | | | | | | | |
| 40° C. ± | 11.07 ± 0.05 | 25.47 ± 0.17 | 53.91 ± 0.07 | N/A | N/A | N/A | N/A |
| 2° C./75% | | | | | | | |
| RH ± 5% | | | | | | | |
| RH | | | | | | | |
| 60° C. Oven | 48.83 ± 0.21 | 146.53 ± 1.39 | 314.28 ± 0.87 | N/A | N/A | N/A | N/A |

### Example 12

39 Months Stability Test Results Summary for Naloxone Hydrochloride Injectable Solution, 100 mg/10 mL (10 mg/ml).

TABLE 12
Stability of Naloxone Solutions 10 mg/mL at 39 Months
Storage Condition 5° C. ± 3° C.

| | | Time Point (months) | | | | |
|---|---|---|---|---|---|---|
| | | 0 | 3 | 6 | 12 | 18 |
| | | Sample Pull Date | | | | |
| Test Name | | N/A | Feb. 14, 2020 | May 16, 2020 | Nov. 13, 2020 | May 15, 2021 |
| & Method | Specification | Results | | | | |
| Appearance, | Clear | Clear | Clear | Clear | Clear | Clear |
| Visual | | | | | | |
| Assay | 90.0-110.0% of | 100.43 | 99.10 | 99.70 | 99.63 | 100.10 |
| | label claim | | | | | |
| Related | Noroxymorphone (A) | A = NP | AA = 0.01 | A = NP | A = 0.01% | A = NP |
| Substances | ≤0.2% | B = NP | B = NP | B = NP | B = NP | B = NP |
| | 3-O-allylnaloxone (B) | C = 0.05% | C = 0.07% | C = 0.05% | C = 0.05% | C = 0.02% |
| | ≤0.2% | D = NP | D = NP | D = NP | D = NP | D = NP |
| | 10-α- | E = NP | E = 0.03% | E = NP | E = 0.01% | E = 0.01% |
| | Hydroxynaloxone (C) | F = NP | F = NP | F = NP | F = NP | F = NP |
| | ≤0.2% | RRT0.51 = | RRT0.51 = | RRT0.51 = | RRT0.51 = 0.01% | RRT0.51 = 0.01% |
| | 7,8- | NP | NP | NP | RRT0.61 = 0.01% | RRT0.61 = 0.01% |
| | didehydronaloxone(D) | RRT0.61 = | RRT0.61 = 0.03% | RRT0.61 = | Unspecified: | Unspecified: |
| | ≤0.3% | NP | Unspecified: | NP | US RRT | US RRT |
| | 2,2-Bisnaloxone (E) | Unspecified: | US RRT | Unspecified: | 0.32 = NP % | 0.32 = NP |
| | ≤0.2% | US RRT | 0.32 = NP | US RRT | US RRT | US RRT |
| | 10-β- | 0.32 = NP | US RRT | 0.32 = NP | 0.58 = NP | 0.58 = NP |
| | Hydroxynaloxone (F) | US RRT | 0.58 = NP | US RRT | US RRT | US RRT |
| | ≤0.2% | 0.58 = NP | US RRT | 0.58 = NP | 0.71 = 0.01% | 0.71 = NP |
| | Specified Related | US RRT | 0.71 = 0.01% | US RRT | Total = 0.05% | Total = 0.32% |
| | Substance at RRT | 0.71 = NP | Total = 0.07% | 0.71 = NP | | |
| | 0.51 | Total = 0.05% | | Total = 0.05% | | |
| | ≤0.35% | | | | | |
| | Specified Related | | | | | |
| | Substance at RRT | | | | | |
| | 0.61 | | | | | |
| | ≤0.35% | | | | | |
| | Unspecified Related | | | | | |
| | Substances (each) | | | | | |
| | ≤0.2% | | | | | |
| | Total Related | | | | | |
| | Substances | | | | | |
| | ≤1.2% | | | | | |
| Preservative | Methylparaben: | 99.75 | 100.67 | 100.43 | 100.37 | 99.97 |
| Content | 90.0-110.0% of | | | | | |
| | label claim | | | | | |
| | Propylparaben: | 100.37 | 100.23 | 100.30 | 101.17 | 100.73 |
| | 90.0-110.0% of | | | | | |
| | label claim | | | | | |
| | 4-hydroxy benzoic | 4.73 | 4.93 | 5.25 | 5.19 | 5.63 |
| | acid ≤75 μg/mL | | | | | |
| pH | 3.0-3.5 | 3.10 | 3.11 | 3.08 | 3.05 | 2.86 |
| Osmolality | 330 to 360 | 357.67 | 355.67 | 355.67 | 359.00 | 356.00 |
| | mOsmol/kg | | | | | |

| | | Time Point (months) | | |
|---|---|---|---|---|
| | | 24 | 36 | 39 |
| | | Sample Pull Date | | |
| Test Name | | Nov. 16, 2021 | Nov. 15, 2022 | Feb. 15, 2023 |
| & Method | Specification | Results | | |
| Appearance, | Clear | Clear | Clear | Clear |
| Visual | | | | |
| Assay | 90.0-110.0% of | 99.90 | 100.3 | 100.3 |
| | label claim | | | |
| Related | Noroxymorphone (A) | A = NP | A = 0.01 | A = 0.02% |
| Substances | ≤0.2% | B = NP | B = NP | B = NP |
| | 3-O-allylnaloxone (B) | C = 0.02% | C = 0.09 | C = 0.01% |
| | ≤0.2% | D = NP | D = NP | D = NP |
| | 10-α- | E = 0.01% | E = 0.01% | E = 0.02% |
| | Hydroxynaloxone (C) | F = NP | F = NP | F = NP |
| | ≤0.2% | RRT0.51 = 0.01% | RRT0.51 = 0.01% | RRT0.51 = 0.05% |
| | 7,8- | RRT0.61 = 0.01% | RRT0.61 = 0.03% | RRT0.61 = 0.06% |
| | didehydronaloxone(D) | Unspecified: | Unspecified: | Unspecified: |
| | ≤0.3% | US RRT | US RRT | US RRT |
| | 2,2-Bisnaloxone (E) | 0.32 = NP | 0.32 = NP | 0.32 = NP |
| | ≤0.2% | US RRT | US RRT | US RRT |
| | 10-β- | 0.58 = NP | 0.58 = NP | 0.58 = NP |
| | Hydroxynaloxone (F) | US RRT | US RRT | US RRT |
| | ≤0.2% | 0.71 = NP | 0.71 = NP | 0.71 = NP |
| | Specified Related | Total = 0.0% | Total = 0.05% | Total = 0.11% |
| | Substance at RRT | | | |
| | 0.51 | | | |
| | ≤0.35% | | | |
| | Specified Related | | | |
| | Substance at RRT | | | |
| | 0.61 | | | |
| | ≤0.35% | | | |
| | Unspecified Related | | | |
| | Substances (each) | | | |
| | ≤0.2% | | | |
| | Total Related | | | |
| | Substances | | | |
| | ≤1.2% | | | |
| Preservative | Methylparaben: | 100.0 | 99.9 | 99.5 |
| Content | 90.0-110.0% of | | | |
| | label claim | | | |
| | Propylparaben: | 100.1 | 99.9 | 99.6 |
| | 90.0-110.0% of | | | |
| | label claim | | | |
| | 4-hydroxy benzoic | 5.84 | 6.99 | 7.33 |
| | acid ≤75 μg/mL | | | |
| pH | 3.0-3.5 | 2.98 | Not tested | Not Tested |
| Osmolality | 330 to 360 | 358.67 | Not tested | Not tested |
| | mOsmol/kg | | | |

Clear = Clear/Colorless

### Example 13

39 Months Stability Test Results Summary for Naloxone Hydrochloride Injectable Solution, 100 mg/10 mL (10 mg/mL).

TABLE 13
Stability of Naloxone Solutions 10 mg/mL at 39 Months
Storage Condition 25° C. ± 2° C./60% RH ± 5% RH

| | | Time Point (months) | | | | |
|---|---|---|---|---|---|---|
| | | 0 | 3 | 6 | 12 | 18 |
| | | Sample Pull Date | | | | |
| Test Name | | N/A | Feb. 14, 2020 | May 16, 2020 | Nov. 13, 2020 | May 15, 2021 |
| & Method | Specification | Results | | | | |
| Appearance, | Clear | Clear | Clear | Clear | Clear | Clear |
| Visual | | | | | | |
| Assay | 90.0-110.0% of | 100.43 | 99.03 | 99.53 | 99.57 | 100.20 |
| | label claim | | | | | |
| Related | Noroxymorphone (A) | A = NP | A = 0.02% | A = NP | A = 0.04% | A = 0.04% |
| Substances | ≤0.2% | B = NP | B = NP | B = NP | B = NP | B = NP |
| | 3-O-allylnaloxone (B) | C = 0.05% | C = 0.08% | C = 0.07% | C = 0.04% | C = 0.05% |
| | ≤0.2% | D = NP | D = NP | D = NP | D = NP | D = NP |
| | 10-α- | E = NP | E = 0.04% | E = 0.05% | E = 0.07% | E = 0.08% |
| | Hydroxynaloxone (C) | F = NP | F = NP | F = NP | F = NP | F = NP |
| | ≤0.2% | RRT0.51 = NP | RRT0.51 = 0.02% | RRT0.51 = NP | RRT0.51 = 0.06% | RRT0.51 = 0.05% |
| | 7,8- | RRT0.61 = NP | RRT0.61 = 0.07% | RRT0.61 = 0.09% | RRT0.61 = 0.15% | RRT0.61 = 0.14% |
| | didehydronaloxone(D) | Unspecified: | Unspecified: | Unspecified: | Unspecified: | Unspecified: |
| | ≤0.3% | US RRT | US RRT | US RRT | US RRT | US RRT |
| | 2,2-Bisnaloxone (E) | 0.32 = NP | 0.32 = 0.01% | 0.32 = NP | 0.32 = 0.02% | 0.32 = NP |
| | ≤0.2% | US RRT | US RRT | US RRT | US RRT | US RRT |
| | 10-β- | 0.58 = NP | 0.58 = NP | 0.58 = NP | 0.58 = NP | 0.58 = NP |
| | Hydroxynaloxone (F) | US RRT | US RRT | US RRT | US RRT | US RRT |
| | ≤0.2% | 0.71 = NP | 0.71 = 0.02% | 0.71 = NP | 0.71 = 0.01% | 0.71 = NP |
| | Specified Related | Total = 0.05% | Total = 0.15% | Total = 0.21% | Total = 0.28% | Total = 0.32% |
| | Substance at RRT | | | | | |
| | 0.51 | | | | | |
| | ≤0.35% | | | | | |
| | Specified Related | | | | | |
| | Substance at RRT | | | | | |
| | 0.61 | | | | | |
| | ≤0.35% | | | | | |
| | Unspecified Related | | | | | |
| | Substances (each) | | | | | |
| | ≤0.2% | | | | | |
| | Total Related | | | | | |
| | Substances | | | | | |
| | ≤1.2% | | | | | |
| Preservative | Methylparaben: | 99.75 | 100.87 | 100.03 | 100.57 | 99.17 |
| Content | 90.0-110.0% of | | | | | |
| | label claim | | | | | |
| | Propylparaben: | 100.37 | 100.50 | 99.13± | 99.83 | 100.00 |
| | 90.0-110.0% of | | | | | |
| | label claim | | | | | |
| | 4-hydroxy benzoic | 4.73 | 8.40 | 14.04 | 19.58 | 25.35 |
| | acid ≤75 μg/mL | | | | | |
| pH | 3.0-3.5 | 3.10 | 3.11 | 3.04 | 3.04 | 2.81 |
| Osmolality | 330 to 360 | 357.67 | 355.67 | 357.33 | 359.00 | 358.00 |
| | mOsmol/kg | | | | | |

| | | Time Point (months) | | |
|---|---|---|---|---|
| | | 24 | 36 | 39 |
| | | Sample Pull Date | | |
| Test Name | | Nov. 16, 2021 | Nov. 15, 2022 | Feb. 15, 2023 |
| & Method | Specification | Results | | |
| Appearance, | Clear | Clear | Clear | Clear |
| Visual | | | | |
| Assay | 90.0-110.0% of | 100.10 | 99.8 | 100.1 |
| | label claim | | | |
| Related | Noroxymorphone (A) | A = 0.05% | A = 0.12 | A = 0.13 |
| Substances | ≤0.2% | B = NP | B = NP | B = NP |
| | 3-O-allylnaloxone (B) | C = 0.02% | C = 0.09 | C = 0.11 |
| | ≤0.2% | D = NP | D = NP | D = NP |
| | 10-α- | E = 0.09% | E = 0.12 | E = 0.12 |
| | Hydroxynaloxone (C) | F = NP | F = NP | F = NP |
| | ≤0.2% | RRT0.51 = 0.07% | RRT0.51 = 0.18% | RRT0.51 = 0.25% |
| | 7,8- | RRT0.61 = 0.14% | RRT0.61 = 0.21% | RRT0.61 = 0.29% |
| | didehydronaloxone(D) | Unspecified: | Unspecified: | Unspecified: |
| | ≤0.3% | US RRT | US RRT | US RRT |
| | 2,2-Bisnaloxone (E) | 0.32 = NP | 0.32 = 0.05% | 0.32 = 0.06% |
| | ≤0.2% | US RRT | US RRT | US RRT |
| | 10-β- | 0.58 = NP | 0.58 = NP | 0.58 = NP |
| | Hydroxynaloxone (F) | US RRT | US RRT | US RRT |
| | ≤0.2% | 0.71 = NP | 0.71 = NP | 0.71 = NP |
| | Specified Related | Total = 0.35% | US RRT | US RRT |
| | Substance at RRT | | 0.79 = 0.05% | 0.79 = 0.06% |
| | 0.51 | | Total = 0.81% | Total = 1.01% |
| | ≤0.35% | | | |
| | Specified Related | | | |
| | Substance at RRT | | | |
| | 0.61 | | | |
| | ≤0.35% | | | |
| | Unspecified Related | | | |
| | Substances (each) | | | |
| | ≤0.2% | | | |
| | Total Related | | | |
| | Substances | | | |
| | ≤1.2% | | | |
| Preservative | Methylparaben: | 98.4 | 97.4 | 97.0 |
| Content | 90.0-110.0% of | | | |
| | label claim | | | |
| | Propylparaben: | 100.27 | 99.6 | 99.5 |
| | 90.0-110.0% of | | | |
| | label claim | | | |
| | 4-hydroxy benzoic | 34.26 | 48.47 | 51.61 |
| | acid ≤75 μg/mL | | | |
| pH | 3.0-3.5 | 2.98 | Not tested | Not Tested |
| Osmolality | 330 to 360 | 359.00 | Not tested | Not tested |
| | mOsmol/kg | | | |

Clear = Clear/Colorless

### Example 14

6 Months Thermostability Test Results Summary for Naloxone Hydrochloride Injectable Solution, 100 mg/10 mL (10 mg/mL).

TABLE 14
6 Month Thermostability Test Results Summary for Naloxone Hydrochloride Injectable
Solution, 100 mg/10 mL Storage Condition 40° C. ± 2° C./75% RH ± 5% RH

| | | Time Pull (months) | | |
|---|---|---|---|---|
| | | 0 | 3 | 6 |
| | | Sample Pull Date | | |
| | | N/A | Feb. 14, 2020 | May 16, 2020 |
| | Test Name & Method | | | |
| | Specification | Result | | |
| Appearance, | Clear, Colorless solution | Clear, Colorless | Clear, Colorless | Clear, Colorless |
| Visual | | | | |
| Assay | 90.0-110.0% of label claim | 100.43 | 98.97 | 98.57 |
| Related | Noroxymorphone (A) ≤ 0.2% | A = NP | A = 0.07% | A = 0.12% |
| Substances | 3-O-allylnaloxone (B) ≤ 0.2% | B = NP | B = NP | B = NP |
| | 10-α-Hydroxynaloxone (C) ≤ 0.2% | C = 0.05% | C = 0.10% | C = 0.11% |
| | 7,8-didehydronaloxone(D) ≤ 0.3% | D = NP | D = NP | D = NP |
| | 2,2-Bisnaloxone (E) ≤ 0.2% | E = NP | E = 0.16% | E = 0.18% |
| | 10-β-Hydroxynaloxone (F) ≤ 0.2% | F = NP | F = NP | F = NP |
| | Specified Related Substance at RRT 0.51 ≤ | RRT0.51 = NP | RRT0.51 = 0.15% | RRT0.51 = 0.30% |
| | 0.35% | RRT0.61 = NP | RRT0.61 = 0.24% | RRT0.61 = 0.28% |
| | Specified Related Substance at RRT 0.61 ≤ | Unspecified: | Unspecified: | Unspecified: |
| | 0.35% | US RRT 0.32 = NP | US RRT 0.32 = 0.03% | US RRT 0.32 = 0.05% |
| | Unspecified Related Substances (each) ≤ 0.2% | US RRT 0.58 = NP | US RRT 0.58 = NP | US RRT 0.58 = NP |
| | Total Related Substances ≤ 1.2% | US RRT 0.71 = NP | US RRT 0.71 = 0.05% | US RRT 0.71 = 0.07% |
| | | Total = 0.05% | Total = 0.77% | Total = 1.11% |
| Preservative | Methylparaben | 99.75 | 100.37 | 97.87 |
| Content | 90.0-110.0% of label claim | | | |
| | Propylparaben: | 100.37 | 99.77 | 99.90 |
| | 90.0-110.0% of label claim | | | |
| | 4-hydroxy benzoic acid ≤ 75 μg/mL | 4.73 | 25.47 | 53.91 |
| pH | 3.0-3.5 | 3.10 | 3.05 | 3.04 |
| Osmolarity | 330 to 360 mOsmol/kg | 357.67 | 354.00 | 356.00 |

### Example 15

### Pharmacokinetic Study in Beagle Dogs

TABLE 15
Pharmacokinetic Parameters in Male and Female Beagle Dogs after a Single
Intravenous or for Intramuscular Dose of Naloxone: Effect of Formulation

| | | Mean | t_{1/2} | Tₘₐₓ | Cₘₐₓ | C₀ | AUCₗₐₛₜ | AUC_{inf} | MRTₗₐₛₜ | Bioavailab |
|---|---|---|---|---|---|---|---|---|---|---|
| Group | Sex | or SD | (hr) | (hr)^{a} | (pg/ml) | (pg/ml) | (hr · pg/ml) | (hr · pg/ml) | (hr) | Hity, F (%,) |
| 1 | Male | Mean | 0.58 | 0.033 | 124000 | 172000 | 47500 | 49300 | 0.51 | NA |
| | | SD | 0.26 | 0.000 | 15000 | 40000 | 11200 | 11300 | 0.16 | NA |
| 1 | Female | Mean | 0.41 | 0.033 | 90500 | 117000 | 36900 | 38100 | 0.43 | NA |
| | | SD | 0.03 | 0.000 | 15600 | 30100 | 5100 | 5400 | O.OI | NA |
| 2 | Mak | Mean | 0.54 | 0.221 | 68900 | NA | 48200 | 53600 | 0.67 | 102 |
| | | SD | 0.12 | 0.094 | 18700 | NA | 3180 | 3650 | 0.16 | 6.7 |
| 2^{b} | Female | Mean | 0.44 | 0.111 | 54000 | NA | 41100 | 39800 | 0.65 | 103 |
| | | SD | NA | 0.048 | 7390 | NA. | 7310 | NA | 0.18 | NA |
| 3 | Male | Mean | 0.40 | 0.165 | 49900 | NA | 31700 | 32900 | 0.53 | 66.7 |
| | | SD | 0.06 | 0.143 | 20400 | NA | 4340 | 4800 | 0.02 | 9.1 |
| 3 | Female | Mean | 0.34 | 0.111 | 47800 | NA | 29700 | 30400 | 0.48 | 80.6 |
| | | SD | 0.04 | 0.048 | 11]00 | NA | 5290 | 5590 | 0.10 | 14.3 |

Group 1 = IV;
Group 2 and Group 3 = IM
NA: not applicable
^{a}median Tₘₐₓ values were: G1 males and females: 0.033 hr, G2 males: 0.167; G2 females, G3 males and females: 0.083 hr
^{b}Dog 12 terminal phase had insufficient linenrity to calculate t_{1/2} AUC_{iof}, and F; N = 2 females of this group

Nonclinical Pharmacokinetics were studied for the disclosed Naloxone formulation. The plasma pharmacokinetics and bioavailability of Naloxone in a modified formulation (Formulation 1) to Naloxone in non-buffered saline solution (Formulation 2, the current formulation for marketed Naloxone products) were compared. Male and female Beagle dogs were administered a single dose of Naloxone at 0.27 mg/kg in the Formulation 1 by IV (Group 1) or IM injection (Group 2) injection or in Formulation 2 by IM injection. The same dogs were used for each dose administration following a wash-out period. Blood was collected from the IV-treated dogs at pre-dose 2, 5, 10, 20, 30 minutes and 1, 2, 4, and 8 hour post-dose for processing to plasma. Also, blood was collected from the IV-treated dogs at pre-dose, 5, 10, 15, 20, 30 minutes, and 1, 2, 4, and 8 hour post-dose for processing to plasma. Clinical observations were performed at pre-dose, immediately following dose administration and at the last blood collection timepoint, and once daily on non-treatment days. In Group 2 (Naloxone in Formulation 1) one IM-treated male dog exhibited slight vocalization immediately post-dose and another male dog had soft stools on Days 6 and 7. In Group 3 (Naloxone in Formulation 2) one IM-treated male dog exhibited slight vocalization immediately post-dose. All other animals were normal throughout the duration of the study.

In male dogs, naloxone plasma concentrations were above the bioanalytical assay lower limit of quantitation (LLOQ, 500 μg/ml) through 4 hr, in the Group 1 (IV route) and Group 2 (IM route) using Formulation 1. In Group 3, IM route with Formulation 2, naloxone was quantifiable through 2 hours, and all samples were less than the LLOQ at 4 and 8 hours. In the female dogs, naloxone concentrations were above the LLOQ at 2 hours in all three groups and the test article concentrations were less than the LLOQ by 4 hours after dose administration. Results of this study are presented in Table 6. Plasma time profiles for naloxone in this study are presented in FIGS. 35 and 36.

In summary, the intramuscular bioavailability using Formulation 1 (drug product candidate) was essentially 100% (102±6.7% for males and 103% for females) while the bioavailability of naloxone in Formulation 2 was markedly lower 66.7±9.1% and 80.6±14.3% for males and females, respectively).

### Example 16

Formulations of 10 mg/mL Naloxone for Intramuscular Injection

TABLE 16
Formulations of 10 mg/mL Naloxone for intramuscular injection

| | Saline/Non-buffered | | 10 mM Citrate Buffer | |
|---|---|---|---|---|
| | Formulation | | | |
| | 16847-11-1 | | 168447-12-1 | |
| | Batch Size | | | |
| | 1200 ml | | 1200 ml | |
| | % w/w | g | % w/w | g |
| Naloxone HCl dihydrate, | 1.000 | 12 | 1.000 | 12.000 |
| USP | | | | |
| Citric acid, monohydrate, | — | — | 0.172 | 2.064 |
| USP | | | | |
| Trisodium citrate | — | — | 0.053 | 0.636 |
| dehydrate, USP | | | | |
| Sodium chloride, USP | 0.900 | 10.800 | 0.900 | 10.80 |
| Methylparaben, NF | 0.180 | 2.1600 | 0.180 | 2.160 |
| Methylparaben, NF | 0.020 | 0.2400 | 0.020 | 0.240 |
| WFI, USP | 97 | q.s. | 97.680 | q.s. |
| Total | 100.000 | 1200.000 | 100.000 | 1200.000 |

### EMBODIMENTS

### Embodiment 1

An aqueous pharmaceutical composition comprising:
(a) naloxone hydrochloride or a pharmaceutically acceptable salt thereof,
(b) one or more preservatives,
(c) one or more buffering agents,
(d) a tonicity modifier, and
(e) optionally a pH modifier.

### Embodiment 2

The aqueous pharmaceutical composition of embodiment 1, wherein the naloxone hydrochloride or pharmaceutically acceptable salt thereof is present at a dose of about 3-4 mg/mL, 4.5-5 mg/mL, 5.5-6.5 mg/mL, 7-8 mg/mL, 8.5-9.5 mg/mL, 10-11 mg/ml, 11.5-12 mg/mL, 13-14 mg/mL, 14.5-15 mg/mL, 15.5-16 mg/ml, 16.5-17 mg/mL, or 17-18 mg/mL.

### Embodiment 3

The aqueous pharmaceutical composition of embodiment 1, wherein the one or more preservatives are methylparaben and propylparaben.

### Embodiment 4

The aqueous pharmaceutical composition of embodiment 3, wherein the one or more preservatives are about 0.1-0.5% w/w methylparaben and about 0.1-0.5% w/w propylparaben.

### Embodiment 5

The aqueous pharmaceutical composition of embodiment 1, wherein the one or more hydroxycarboxylic acid buffering agents are trisodium citrate dihydride and citric acid monohydrate.

### Embodiment 6

The aqueous pharmaceutical composition of embodiment 5, wherein the one or more hydroxycarboxylic acid buffering agents are about 0.01-0.1% w/w trisodium citrate dihydride and about 0.01-0.5% w/w citric acid monohydrate.

### Embodiment 7

The aqueous pharmaceutical composition of embodiment 1, wherein the tonicity modifier is sodium chloride, sorbitol, or mannitol.

### Embodiment 8

The aqueous pharmaceutical composition of embodiment 1, wherein the pH modifier is sodium hydroxide or hydrochloric acid.

### Embodiment 9

The aqueous pharmaceutical composition of embodiment 1, wherein the pH of the aqueous pharmaceutical composition is about 1-5.

### Embodiment 10

The aqueous pharmaceutical composition of embodiment 1, wherein intramuscular bioavailability is about 60-100% after injection into a subject.

### Embodiment 11

The aqueous pharmaceutical composition of embodiment 1, wherein the aqueous pharmaceutical composition comprises less than about 0.05-0.15% w/w noroxymorphone as an impurity after storage for six months at 40° C.±2° C. and 75% 5% relative humidity.

### Embodiment 12

In another embodiment, the aqueous pharmaceutical composition of embodiment 1, wherein the aqueous pharmaceutical composition comprises less than about 0.10-0.14% w/w noroxymorphone as an impurity after storage for thirty six months at 25° C.±2° C./60%±5% relative humidity.

### Embodiment 13

The aqueous pharmaceutical composition of embodiment 1, wherein the aqueous pharmaceutical composition comprises less than about 0.01-0.0% w/w noroxymorphone as an impurity after storage for thirty six months at 5° C.±3° C.

### Embodiment 14

The aqueous pharmaceutical composition of embodiment 1, wherein the aqueous pharmaceutical composition is packaged in a single-use vial.

### Embodiment 15

The aqueous pharmaceutical composition of embodiment 1, wherein the aqueous pharmaceutical composition is packaged in a multi-use vial allowing for up to nine repeat doses or about 3-4 mg, 4.5-5 mg, 5.5-6.5 mg, 7-8 mg, 8.5-9.5 mg, 10-11 mg, 11.5-12 mg, 13-14 mg, 14.5-15 mg, 15.5-16 mg, 16.5-17 mg, or 17-18 mg per dose.

### Embodiment 16

The aqueous pharmaceutical composition of either embodiment 13 or embodiment 14, wherein the aqueous pharmaceutical composition is packaged in a clear vial.

### Embodiment 17

The aqueous pharmaceutical composition of either embodiment 13 or embodiment 14, wherein the aqueous pharmaceutical composition is packaged in an opaque container so as to protect from photo-degradation.

### Embodiment 18

An aqueous pharmaceutical composition comprising:
(a) naloxone hydrochloride or a pharmaceutically acceptable salt thereof,
(b) methylparaben,
(c) propylparaben,
(d) trisodium citrate dihydride,
(e) citric acid monohydrate,
(f) a tonicity modifier, and
(g) optionally a pH modifier,
wherein the aqueous pharmaceutical composition has a pH of about 3.0.

### Embodiment 19

The aqueous pharmaceutical composition of embodiment 18, wherein the naloxone hydrochloride or pharmaceutically acceptable salt thereof is present at a dose of about 8.5-9.5 mg/mL, 10-11 mg/ml, 11.5-12 mg/mL, 12-13 mg/ml, 13-14 mg/ml, 14.5-15 mg/mL, 15.5-16 mg/mL, 16.5-17 mg/mL, or 17-18 mg/mL.

### Embodiment 20

The aqueous pharmaceutical composition of embodiment 18, wherein the tonicity modifier is sodium chloride, sorbitol, or mannitol.

### Embodiment 21

The aqueous pharmaceutical composition of embodiment 18, wherein the pH modifier is sodium hydroxide or hydrochloric acid.

### Embodiment 22

The aqueous pharmaceutical composition of embodiment 18, wherein intramuscular bioavailability is about 60-100% after injection into a subject.

### Embodiment 23

The aqueous pharmaceutical composition of embodiment 18, wherein the aqueous pharmaceutical composition comprises less than about 0.05-0.15% w/w noroxymorphone as an impurity after storage for six months at 40° C.±2° C. and 75%±5% relative humidity.

### Embodiment 24

In another embodiment, the aqueous pharmaceutical composition of embodiment 18, wherein the aqueous pharmaceutical composition comprises less than about 0.10-0.14% w/w noroxymorphone as an impurity after storage for thirty six months at 25° C.±2° C./60%±5% relative humidity.

### Embodiment 25

The aqueous pharmaceutical composition of embodiment 18, wherein the aqueous pharmaceutical composition is packaged in a single-use vial.

### Embodiment 26

The aqueous pharmaceutical composition of embodiment 18, wherein the aqueous pharmaceutical composition is packaged in a multi-use vial allowing for up to nine repeat doses of 7-8 mg, 8.5-9.5 mg, 10-11 mg, 11.5-12 mg, 13-14 mg, 14.5-15 mg, 15.5-16 mg, 16.5-17 mg, or 17-18 mg per dose.

### Embodiment 27

The aqueous pharmaceutical composition of either embodiment 25 or embodiment 26, wherein the composition is packaged in a clear vial.

### Embodiment 28

The aqueous pharmaceutical composition of either embodiment 25 or embodiment 26, wherein the composition is packaged in an opaque container so as to protect from photo-degradation.

### Embodiment 29

A parenteral pharmaceutical composition comprising:
(a) naloxone hydrochloride or a pharmaceutically acceptable salt thereof,
(b) methylparaben,
(c) propylparaben,
(d) trisodium citrate dihydride,
(e) citric acid monohydrate,
(f) sodium chloride, and
(g) optionally a pH modifier;
wherein the parenteral pharmaceutical composition has a pH of about 3.0;
and
wherein the naloxone hydrochloride or pharmaceutically acceptable salt thereof is present at a dose of about 10 mg/mL.

### Embodiment 30

The parenteral pharmaceutical composition of embodiment 29, wherein the pH modifier is sodium hydroxide or hydrochloric acid.

### Embodiment 31

The parenteral pharmaceutical composition of embodiment 29, wherein intramuscular bioavailability is about 60-100% after injection into a subject.

### Embodiment 32

The parenteral pharmaceutical composition of embodiment 29 wherein the parenteral pharmaceutical composition comprises less than about 0.05-0.15% w/w noroxymorphone as an impurity after storage for six months at 40° C.±2° C. and 75%±5% relative humidity.

### Embodiment 33

In another embodiment, the aqueous pharmaceutical composition of embodiment 29, wherein the aqueous pharmaceutical composition comprises less than about 0.10-0.14% w/w noroxymorphone as an impurity after storage for thirty six months at 25° C.±2° C./60%±5% relative humidity.

### Embodiment 34

The parenteral pharmaceutical composition of embodiment 29, wherein the parenteral pharmaceutical composition is packaged in a single use vial or auto-injector.

### Embodiment 35

The parenteral pharmaceutical composition of embodiment 29, wherein the parenteral pharmaceutical composition is packaged in a multi-use vial allowing for up to nine repeat doses of 10 mg per dose.

### Embodiment 36

The parenteral pharmaceutical composition of either embodiment 34 or embodiment 35, wherein the parenteral pharmaceutical composition is packaged in a clear vial.

### Embodiment 37

The parenteral pharmaceutical composition of either embodiment 34 or embodiment 35, wherein the parenteral pharmaceutical composition is packaged in an opaque container so as to protect from photo-degradation.

### Embodiment 38

A method of treating ultra-potent synthetic opioid overdose by administering up to nine repeat doses of a parenteral pharmaceutical composition, wherein the parenteral pharmaceutical comprises:
(a) about 1.0% w/w naloxone hydrochloride or a pharmaceutically acceptable salt thereof,
(b) about 0.18% w/w methylparaben,
(c) about 0.02% w/w propylparaben,
(d) about 0.05% w/w trisodium citrate dihydride,
(e) about 0.17% w/w citric acid monohydrate.
(f) about 0.9% w/w sodium chloride, and
(g) optionally a pH modifier of sodium hydroxide or hydrochloric acid;
wherein the parenteral pharmaceutical composition has a pH of about 3.0; and
wherein the naloxone hydrochloride or pharmaceutically acceptable salt thereof is present at a dose of about 10 mg/mL.

### Embodiment 39

The method of embodiment 38, wherein the parenteral pharmaceutical composition is administered intravenously.

### Embodiment 40

The method of embodiment 38, wherein the parenteral pharmaceutical composition is administered by intramuscular injection.

### Embodiment 41

The method of embodiment 38, wherein the parenteral pharmaceutical composition is administered by subcutaneous injection.

### Embodiment 42

A parenteral pharmaceutical composition for the treatment of ultra-potent synthetic opioid overdose, wherein the parenteral pharmaceutical comprises:
(a) about 1.0% w/w naloxone hydrochloride or a pharmaceutically acceptable salt thereof,
(b) about 0.18% w/w methylparaben,
(b) about 0.02% w/w propylparaben,
(d) about 0.05% w/w trisodium citrate dihydride,
(e) about 0.17% w/w citric acid monohydrate,
(f) about 0.9% w/w sodium chloride, and
(g) optionally a pH modifier of sodium hydroxide or hydrochloric acid;
wherein the parenteral pharmaceutical composition has a pH of about 3.0;
and
wherein the naloxone hydrochloride or pharmaceutically acceptable salt thereof is present at a dose of about 10 mg/mL.

### Embodiment 43

Use of a parenteral pharmaceutical composition for the manufacture of a medicament for treating ultra-potent synthetic opioid overdose, wherein the parenteral pharmaceutical comprises:
(a) about 1.0% w/w naloxone hydrochloride or a pharmaceutically acceptable salt thereof,
(b) about 0.18% w/w methylparaben.
(c) about 0.02% w/w propylparaben,
(d) about 0.05% w/w trisodium citrate dihydride,
(e) about 0.17% w/w citric acid monohydrate,
(f) about 0.9% w/w sodium chloride, and
(g) optionally a pH modifier of sodium hydroxide or hydrochloric acid;
wherein the parenteral pharmaceutical composition has a pH of about 3.0;
and
wherein the naloxone hydrochloride or pharmaceutically acceptable salt thereof is present at a dose of about 10 mg/mL.

### Embodiment 44

A kit for administering the aqueous pharmaceutical composition of embodiment 1, the kit comprising:
(a) a testing apparatus to determine if a subject is suffering from opioid poisoning;
(b) an autoinjector, preloaded with a therapeutic amount of the aqueous pharmaceutical composition of claim 1;
(c) a product use pamphlet, describing how to administer and interpret results from the testing apparatus and how to actuate the autoinjector
(d) an opaque container to house the testing apparatus, the autoinjector, and the product use pamphlet,
wherein a subject suffering from opioid poisoning or a first responder treating a subject suffering from opioid poisoning can open the opaque container, read the product use pamphlet, administer the testing apparatus, and upon confirming that the subject is suffering from opioid poisoning, actuate the autoinjector to deliver the pharmaceutical composition of claim 1 to the subject.

### Embodiment 45

The Kit of embodiment 44, wherein the autoinjector, preloaded with a therapeutic amount of the aqueous pharmaceutical composition of embodiment 1 further comprises
(a) about 1.0% w/w naloxone hydrochloride or a pharmaceutically acceptable salt thereof,
(b) about 0.18% w/w methylparaben,
(c) about 0.02% w/w propylparaben,
(d) about 0.05% w/w trisodium citrate dihydride,
(e) about 0.17% w/w citric acid monohydrate,
(f) about 0.9% w/w sodium chloride, and
(g) optionally a pH modifier of sodium hydroxide or hydrochloric acid;
wherein the aqueous pharmaceutical composition has a pH of about 3.0; and
wherein the naloxone hydrochloride or pharmaceutically acceptable salt thereof is present at a dose of about 10 mg/mL.

It is understood that the examples and embodiments described herein are for illustrative purposes only and that various modifications or changes in light thereof will be suggested to persons skilled in the art and are to be incorporated within the spirit and purview of this application and scope of the appended claims.

1. An aqueous pharmaceutical composition comprising:
(a) naloxone hydrochloride or a pharmaceutically acceptable salt thereof,
(b) one or more preservatives,
(c) one or more hydroxycarboxylic acid buffering agents,
(d) a tonicity modifier, and
(e) a pH modifier, wherein the pH of the aqueous pharmaceutical composition is about 1-5.

2. The aqueous pharmaceutical composition of claim 1, wherein the naloxone hydrochloride or pharmaceutically acceptable salt thereof is present at a dose of about 3-4 mg/mL, 4.5-5 mg/mL, 5.5-6.5 mg/mL, 7-8 mg/ml, 8.5-9.5 mg/mL, 10-11 mg/mL, 11.5-12 mg/mL, 13-14 mg/mL, 14.5-15 mg/mL, 15.5-16 mg/mL, 16.5-17 mg/mL, or 17-18 mg/mL.

3. The aqueous pharmaceutical composition of claim 1, wherein the one or more preservatives are methylparaben and propylparaben.

4. (canceled)

5. The aqueous pharmaceutical composition of claim 1, wherein the one or more hydroxycarboxylic acid buffering agents are trisodium citrate dihydride and citric acid monohydrate.

6. (canceled)

7. The aqueous pharmaceutical composition of claim 1, wherein the tonicity modifier is sodium chloride, sorbitol, or mannitol.

8. The aqueous pharmaceutical composition of claim 1, wherein the pH modifier is sodium hydroxide or hydrochloric acid.

9. (canceled)

10. The aqueous pharmaceutical composition of claim 1, wherein intramuscular bioavailability is about 60-100% after injection into a subject.

11. The aqueous pharmaceutical composition of claim 1, wherein the aqueous pharmaceutical composition comprises less than about 0.05-0.15% w/w noroxymorphone as an impurity after storage for six months at 40° C.±2° C. and 75%±5% relative humidity.

12. The aqueous pharmaceutical composition of claim 1, wherein the aqueous pharmaceutical composition comprises less than about 0.10-0.14% w/w noroxymorphone as an impurity after storage for thirty six months at 25° C.±2° C./60%±5% relative humidity.

13. The aqueous pharmaceutical composition of claim 1, wherein the aqueous pharmaceutical composition comprises less than about 0.01-0.0% w/w noroxymorphone as an impurity after storage for thirty six months at 5° C.±3° C.

14. The aqueous pharmaceutical composition of claim 1, wherein the aqueous pharmaceutical composition is packaged in a single use vial or an auto-injector.

15. The aqueous pharmaceutical composition of claim 1, wherein the aqueous pharmaceutical composition is packaged in a multi-use vial allowing for more than one dose of about 3-4 mg, 4.5-5 mg, 5.5-6.5 mg, 7-8 mg, 8.5-9.5 mg, 10-11 mg, 11.5-12 mg, 13-14 mg, 14.5-15 mg, 15.5-16 mg, 16.5-17 mg, or 17-18 mg per dose.

16. (canceled)

17. The aqueous pharmaceutical composition of claim 1, wherein the aqueous pharmaceutical composition is packaged in an opaque container so as to protect from photo-degradation.

18. An aqueous or parenteral pharmaceutical composition comprising:
(a) naloxone hydrochloride or a pharmaceutically acceptable salt thereof,
(b) methylparaben,
(c) propylparaben,
(d) trisodium citrate dihydride,
(e) citric acid monohydrate,
(f) a tonicity modifier, and
(g) a pH modifier, wherein the aqueous or parenteral pharmaceutical composition has a pH of about 3.0.

19. The aqueous or parenteral pharmaceutical composition of claim 18, wherein the naloxone hydrochloride or pharmaceutically acceptable salt thereof is present at a dose of about 8.5-9.5 mg/mL, 10-11 mg/mL, 11.5-12 mg/mL, 12-13 mg/mL, 13-14 mg/mL, 14.5-15 mg/mL, 15.5-16 mg/mL, 16.5-17 mg/mL, or 17-18 mg/mL.

20.-24. (canceled)

25. The aqueous or parenteral pharmaceutical composition of claim 18, wherein the aqueous or parenteral pharmaceutical composition is packaged in a single-use vial.

26. The aqueous or parenteral pharmaceutical composition of claim 18, wherein the aqueous or parenteral pharmaceutical composition is packaged in a multi-use vial allowing for up to nine repeat doses of 7-8 mg, 8.5-9.5 mg, 10-11 mg, 11.5-12 mg, 13-14 mg, 14.5-15 mg, 15.5-16 mg, 16.5-17 mg, or 17-18 mg per dose.

27.-37. (canceled)

38. A method of treating ultra-potent synthetic opioid overdose by administering one or more repeat doses of a parenteral pharmaceutical composition, wherein the parenteral pharmaceutical comprises:
(a) about 1.0% w/w naloxone hydrochloride or a pharmaceutically acceptable salt thereof,
(b) about 0.18% w/w methylparaben,
(c) about 0.02% w/w propylparaben,
(d) about 0.05% w/w trisodium citrate dihydride,
(e) about 0.17% w/w citric acid monohydrate,
(f) about 0.9% w/w sodium chloride, and
(g) a pH modifier of sodium hydroxide or hydrochloric acid;
wherein the parenteral pharmaceutical composition has a pH of about 3.0; and
wherein the naloxone hydrochloride or pharmaceutically acceptable salt thereof is present at a dose of about 10 mg/mL.

39. The method of claim 38, wherein the parenteral pharmaceutical composition is administered intravenously.

40. The method of claim 38, wherein the parenteral pharmaceutical composition is administered by intramuscular injection.

41. The method of claim 38, wherein the parenteral pharmaceutical composition is administered by subcutaneous injection.

42.-45. (canceled)
